(19) United States
(12) Patent Application Publication
(10) Pub. No.: US 2024/0041976 A1
(43) Pub. Date: Feb. 8, 2024

(54) COMPOSITION AND USE

(71) Applicants: Norwegian University of Life Sciences, Ås (NO); Norwegian Institute of Bioeconomy Research, Ås (NO)

(72) Inventors: Dzung DIEP, Ås (NO); Kirill OVCHINNIKOV, Ås (NO); Christian KRANJEC, Ås (NO); Tage THORSTENSEN, Ås (NO)

(73) Assignees: Norwegian University of Life Sciences, Ås (NO); Norwegian Institute of Bioeconomy Research, Ås (NO)

(21) Appl. No.: 18/026,008

(22) PCT Filed: Sep. 14, 2021
(86) PCT No.: PCT/EP2021/075210

§ 371 Date: Mar. 13, 2023

(30) Foreign Application Priority Data
Sep. 14, 2020 (GB) 2014427.5

(51) Int. Cl. A61K 38/16; A61K 31/045; A61K 31/65; A61K 31/165; A61K 31/496; A61K 31/7036; A61K 31/43; A61P 31/04

(52) U.S. Cl. CPC A61K 38/164; A61K 31/045; A61K 31/65; A61K 31/165; A61K 31/496; A61K 31/7036; A61K 31/43; A61P 31/04

(57) ABSTRACT

The invention relates to antibacterial compositions comprising micrococcin P1 and at least one additional antibacterial agent, which is preferably a bacteriocin or an antibiotic. The compositions may be used as an antibacterial, particularly for preventing or treating a bacterial infection.

The present invention relates to an antibacterial composition comprising micrococcin P1 and at least one additional antibacterial agent. This composition is particularly useful in treating a variety of bacterial infections, particularly drug-resistant strains of bacteria.

The spread of antibiotic-resistant bacteria poses a great threat to public health and is getting worse as the current progress in developing new antibiotics is limited (Brown & Wright, 2016, Nature, Vol. 529, p 336-343). Aside from the introduction of carbapenems in 1985, all new antibiotics between the early 1960s and 2000 were synthetic derivatives of existing scaffolds, which often allow resistant strains to arise rapidly (Fischbach & Walsh, 2009, Science, Vol. 325, p 1089-1093). In the USA alone, the economic loss incurred by antibiotic resistance is estimated to be up to 55 billion USD per year with some data suggesting that the total cost may be even higher (Smith & Coast, 2013, BMJ, Vol. 346, f1493). Staphylococcus bacteria are particularly problematic and although staphylococci live as harmless colonizers, when the skin barrier becomes impaired or in high-risk individuals (i.e. immunosuppressed individuals) they can become important opportunistic pathogens. Among these, Staphylococcus aureus (S. aureus) is considered to be the most pathogenic, being associated with a range of clinical conditions: from self-remissive skin infections to life threatening syndromes (Kim et al., 2018, BMC Infect. Dis., Vol. 18, p 86). The main reason for this is the high propensity of S. aureus to acquire resistance to antibiotics.

The formation of biofilms represents a major therapeutic complication and, in some cases, makes the bacterial infection untreatable leaving the surgical removal of the infected area the sole therapeutic option (Tong et al., 2015, Clin. Microbiol. Rev., Vol. 28, p 603-661). It has been estimated that about 80% of all chronic infections are associated with biofilm formation (Jamal et al., 2018, J. Chin. Med. Assoc., Vol. 81, p 7-11) and, among Gram-positive bacteria, S. aureus infections are among those with the highest association with biofilms (Costerton et al., 1999, Science, Vol. 284, p 1318-1322; Lynch and Robertson, 2008, Annu. Rev. Med., Vol. 59, p 415-428).

Consequently, there is a need for new antimicrobials that can be used as alternatives to conventional antibiotics, particularly to treat antibiotic-resistant bacteria, particularly when present in biofilms.

Many antimicrobials, including antibiotics and antimicrobial peptides from eukaryotes and prokaryotes, have been explored and exploited for their therapeutic potential. Antibiotics have been widely exploited in human and animal medicine. However, due to overuse and misuse of antibiotics many pathogens have become resistant to most if not all antibiotics, leaving only a few treatment options.

One alternative to antibiotics is antibacterial peptides (bacteriocins), which can be used in both the food industry and in medicine (Cotter et al., 2013, Nature Reviews. Microbiology, Vol. 11, p 95-105; Cleveland et al., 2001, Int. J. Food Microbiol., Vol. 71, p 1-20; O'Sullivan et al., 2002, Biochimie, Vol. 84, p 593-604).

Bacteriocins are ribosomally synthesized antimicrobial peptides produced by many different bacteria (Cotter et al., 2005, Nature Reviews Microbiology, Vol. 3, p 777-788; Diep and Nes, 2002, Current Drug Targets, Vol. 3, p 107-122). Their antimicrobial activity is generally targeting only towards species/genera closely related to the producer (Nissen-Meyer and Nes, 1997, Archives of Microbiol., Vol. 167, p 67-77), though some can have broader inhibition spectra including food spoilage and pathogenic microorganisms (Gillor and Ghazaryan, 2007, Recent Patents on Anti-infective Drug Discovery, Vol. 2, p 115-122). Bacteriocins bind to specific receptors on target membranes to kill cells. Usually the bacteriocin producer is protected against its own bacteriocin by the immunity protein whose gene is located at the same operon as the structural gene of its corresponding bacteriocin (Diep and Nes, 2002, supra).

Micrococcin P1 (MP1) is a heavily modified bacteriocin belonging to the group thiopeptides. MP1 kills target cells by blocking protein synthesis. It has a very potent activity (MIC at nM concentrations) toward many gram-positive bacteria/pathogens. MP1 has therefore been considered as having potential in treating infections. Whilst MP1 has been regarded as a promising therapeutic alternative to antibiotics against S. aureus MRSA and mycobacterial infections (Akasapu et al., 2019, Chem. Sci., Vol. 10, p 1971-1975; Degiacomi et al., 2016, Tuberculosis (Edinb), Vol. 100, p 95-101), resistance towards MP1 is frequent, hence hampering the use of this bacteriocin.

It has now surprisingly been found that MP1 can act as an additive to improve the activity of other antibacterials against Gram-positive infections. It has been found that MP1 acts synergistically with other antimicrobial agents irrespective of their nature or mode of action; these include other bacteriocins, antibiotics and essential oils, for example. The addition of MP1 to antibacterial formulations has been shown to increase the potency of those formulations against important human pathogens of the genera Enterococcus and Staphylococcus, including their respective drug-resistant variants VRE and MRSA.

In particular, the inventors have found that combinations including MP1 were effective against a large panel of Staphylococcus aureus strains including several MRSA clinical isolates. The compositions were found effective in both killing MRSA cells at a wound site in a murine infection model as well as MRSA cells in biofilms (see the Examples). Efficacy against other bacterial strains (including drug-resistant variants) and using various antibacterials in the composition has also been observed. It appears that MP1 may act to re-sensitize bacteria cells to antibacterials (such as penicillin) and thus can revitalise antibiotics that have been rendered useless due to antibiotic resistance.

The addition of MP1 thus offers a simple and cost-effective strategy to improve the activity of existing antibacterials. Benefits associated with the combinatorial use of MP1 include an increased spectrum of antimicrobial activity, prevention of drug resistance development (lower risk of therapeutic failure) and overall reduction of the antimicrobial dosage (reduction of costs and of potential side effects). Furthermore, the adjuvant use of MP1 allows the efficacy of existing antimicrobial agents to be increased such that available technologies can be rapidly implemented thereby reducing time and costs linked to the de novo development of antibacterial molecules.

Thus, in a first aspect the present invention provides an antibacterial composition comprising micrococcin P1 and at least one additional antibacterial agent.

Micrococcin P1 (MP1) has the structure shown below and is available commercially.

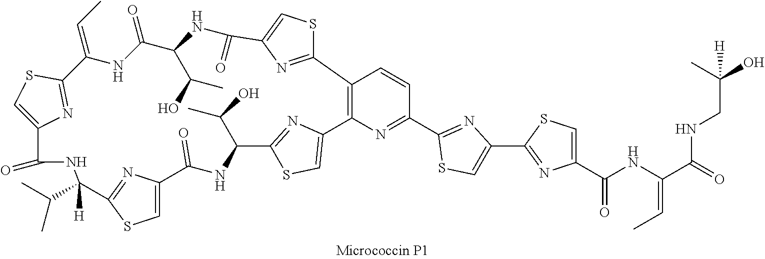 [SMILES: C/C=C(\NC(=O)c1csc(-c2csc(-c3ccc4c(n3)-c3csc(n3)[C@H]([C@@H](C)O)NC(=O)c3csc(n3)[C@H](C(C)C)NC(=O)c3csc(n3)/C(=C/C)NC(=O)[C@H]([C@@H](C)O)NC(=O)c3csc-4n3)n2)n1)C(=O)NC[C@@H](C)O]

In compositions as described herein, MP1 may be present at a concentration in the range of 0.001-1 mg/ml or 0.01-100 mg/ml (e.g. 0.01 to 1 mg/ml) in the composition, e.g. for use in in vitro or in vivo methods. In a preferred aspect, for use in vivo, a concentration of 0.01 to 1 mg/ml is used, e.g. 0.1 to 0.3 mg/ml.

An “antibacterial agent” is a molecule or composition that has antibacterial activity. As used herein “antibacterial activity” refers to the ability of the antibacterial agent to kill, damage or prevent the replication of selected bacteria under in vitro conditions, e.g. as set forth in the Examples. The bacteria are preferably as described hereinafter. (As referred to herein bacteria are referred to in both the singular and plural. In particular they are referred to in the singular when defining the type of bacteria to be targetted (i.e. the type, e.g. species, applicable to each bacterium) and in the plural when referring to the treatment to which they may be subjected (i.e. treatment of multiple microorganisms).) “Kill” refers to destruction of a bacteria to the extent that no further replication can take place. “Damage” refers to affecting the bacteria's ability to function normally, such that it may die or be unable to replicate. “Preventing replication” refers to prevention of the replication of the bacteria partially or completely, e.g. according to the percentages described hereinafter. Preferably a method, treatment or use described herein results in the death or damage of at least 25, 50, 75 or 90% of the bacteria to which the treatment is applied or prevents replication such that a bacterial infection is prevented or reduced, e.g. by at least 30, 40, 50, 60, 70, 80 or 90% relative to a control to which the treatment is not applied. In particular, the antibacterial activity may be assessed by determining the MIC value against one or more bacteria. The bacteria to be tested may be selected from a strain of bacteria selected from the genera Staphylococcus, Enterococcus, Lactococcus or Listeria. For the assessment of antibacterial activity the strain is preferably one which has not developed resistance to the antibacterial agent. An antibacterial agent as described herein, with antibacterial activity preferably has a MIC value of less than 500 nM, preferably less than 300 nM, especially preferably less than 100 nM, preferably against one or more strains from the genera Staphylococcus, Enterococcus, Lactococcus or Listeria, preferably from the species Staphylococcus aureus. Preferably said antibacterial agent has a MIC value of less than 100, 75, 30, 20, 10, 5 or 2 μg/ml for a bacteria as described above, when used alone. This measure is used to identify antibacterial agents that may be used in the invention. However, it will be appreciated that these antibacterial agents may not have substantial antibacterial activity against resistant bacteria and for this reason would be used with the MP1 of the invention.

The combination of MP1 and the at least one additional antibacterial agent preferably results in synergy, i.e. the antibacterial activity is greater than the sum of the activity of the individual components.

In a further preferred aspect, the additional antibacterial agent is not a thiopeptide. As noted above, MP1 is a thiopeptide antibiotic. Thiopeptide antibiotics are thiazolyl peptides produced by bacteria and include thiostrepton, cyclothiazomycin, nosiheptide and lactocillin. Advantageously an antibacterial agent with a different mechanism of action is used.

Bacteriocin as the Antibacterial Agent

In a preferred aspect the antibacterial agent is a bacteriocin (i.e. a second bacteriocin relative to the first bacteriocin, MP1). More than one such additional bacteriocin may be used in compositions of the invention.

A “bacteriocin” as described herein, is a proteinaceous or peptidic toxin produced by a bacteria to inhibit the growth of similar or closely related bacterial strain(s). The bacteriocin has antibacterial activity as described hereinbefore. In a preferred aspect, the bacteriocin is produced by any bacteria belonging to the phylum Firmicutes. In a preferred aspect the bacteriocin is produced by a species of Lactococcus (preferably from Lactococcus garvieae), Bacillus or Staphylococcus. Preferably the bacteriocin has antibacterial activity against one or more species of bacteria from the genera Enterococcus, Listeria, Bacillus and Staphylococcus. Preferred second bacteriocins include bacteriocins from Lactococcus garvieae such as Garviecin L1-5, Garvicin ML, Garvieacin Q and Garvicin A, and particularly preferably Garvicin KS. Also preferred are single or multi-peptide bacteriocins from Bacillus (such as Cerein H, Cerein V, Cerein X, as described hereinafter) and Staphylococcus (such as Aureocin A70). Other second bacteriocins that may be used include nisin and pediocin PA-1. Other preferred bacteriocins are as described hereinafter and include EntK1, EntEJ97short, EntEJ97, K1-EJ and EJ-K1.

In a preferred aspect, the second bacteriocin is a peptide (or contains one or more peptides, e.g. in a complex of one or more peptides). In a preferred aspect the bacteriocin is a single peptide or a multi-peptide bacteriocin complex, e.g. a di-, tri- or tetra-peptide complex. As referred to herein a “peptide” is a polymer comprising at least 15 amino acids, preferably at least 25 or 30 amino acids. Preferably the peptide (or each peptide in the complex) contains less than 50, e.g. less than 45, 40 or 35 amino acids e.g. from 35 to 45 amino acids. In one preferred aspect the bacteriocin is not a naturally occurring molecule (e.g. its sequence may be modified or it may be made up of one or more amino acids that are not naturally occurring). The amino acids making up the peptide may be natural L or D amino acids (preferably L amino acids). Alternatively, one or more non-naturally occurring amino acids may be present in the peptides. Such non-naturally occurring amino acids are derivatives of naturally occurring amino acids and include alkyl (e.g. methyl), nor and aminoalkyl derivatives. Appropriate derivatives are selected to maintain functionality.

Bacteriocin peptides for use according to the invention (including MP1) also include those which are modified without affecting the sequence of the peptide, e.g. by chemical modification, including by deglycosylation or glycosylation. Such peptides may be prepared by post-synthesis/isolation modification of the peptide without affecting functionality, e.g. certain glycosylation, methylation etc. of particular residues. The bacteriocin peptides for use according to the invention (as well as MP1) may also take the form of peptidomimetics which may be considered derivatives in which the functional features of the peptide are retained but are presented in the context of a different, e.g. non-peptide structure. Such peptidomimetics have successfully been developed and used in the art, particularly for medical applications. Peptidomimetics, particularly non-peptidic molecules may be generated through various processes, including conformational-based drug design, screening, focused library design and classical medicinal chemistry. Not only may oligomers of unnatural amino acids or other organic building blocks be used, but also carbohydrates, heterocyclic or macrocyclic compounds or any organic molecule that comprises structural elements and conformation that provides a molecular electrostatic surface that mimics the same properties of the 3-dimensional conformation of the peptide may be used and prepared by methods known in the art.

Thus the peptidomimetics may bear little or no resemblance to a peptide backbone. Peptidomimetics may comprise an entirely synthetic non-peptide form (e.g. based on a carbohydrate backbone with appropriate substituents) or may retain one or more elements of the peptide on which it is based, e.g. by derivatizing one or more amino acids or replacing one or more amino acids with alternative non-peptide components. Peptide-like templates include pseudopeptides and cyclic peptides. Structural elements considered redundant for the function of the peptide may be minimized to retain a scaffold function only or removed where appropriate.

When peptidomimetics retain one or more peptide elements, i.e. more than one amino acid, such amino acids may be replaced with a non-standard or structural analogue thereof. Amino acids retained in the sequences may also be derivatised or modified (e.g. labelled, glycosylated or methylated) as long as the functional properties of the bacteriocin peptides (and M P1) for use according to the invention are retained. The peptidomimetics are referred to as being “derivable from” a certain peptide sequence. By this it is meant that the peptidomimetic is designed with reference to a defined peptide sequence, such that it retains the structural features of the peptide which are essential for its function. This may be the particular side chains of the peptide, or hydrogen bonding potential of the structure. Such features may be provided by non-peptide components or one or more of the amino acid residues or the bonds linking said amino acid residues of the peptide may be modified so as to improve certain functions of the peptide such as stability or protease resistance, while retaining the structural features of the peptide which are essential for its function.

Examples of non-standard or structural analogue amino acids which may be used are D amino acids, amide isosteres (such as N-methyl amide, retro-inverse amide, thioamide, thioester, phosphonate, ketomethylene, hydroxymethylene, fluorovinyl, (E)-vinyl, methyleneamino, methylenethio or alkane), L-N methylamino acids, D-α methylamino acids and D-N-methylamino acids.

The peptides also include derivatives which have been modified, e.g. to facilitate their use in various applications, e.g. pharmaceutical applications (discussed below), e.g. by the addition of targeting or functional groups, e.g. to improve lipophilicity, aid cellular transport, solubility and/or stability. Thus oligosaccharides, fatty acids, fatty alcohols, amino acids, peptides or polypeptides may be conjugated to the aforementioned peptides.

The peptides also encompass derivatives in the form of “pro-drugs” or “pro-peptides” such that the added component may be removed by cleavage once administered, e.g. by cleavage of a substituent added through esterification which may be removed by the action of esterases. Such pro-drugs include native precursors of the naturally occurring peptides which are cleaved e.g. by proteolysis to yield the peptide of interest. Such precursors may be inactive in the precursor form but may be activated by proteolytic cleavage.

The table below provides details of the peptide sequences disclosed in the application.

| | | SEQ |
|---|---|---|
| | | ID |
| Name | Sequence | NO: |
| EntK1 | MKFKFNPTGTIVKKLTQYEIAWFKNKHGYYPWEIPRC | 1 |
| EntEJ97 | MLAKIKAMIKKFPNPYTLAAKLTTYEINWYKQQYGRYPWERPVA | 2 |
| EntEJ97short | MIKKFPNPYTLAAKLTTYEINWYKQQYGRYPWERPVA | 3 |
| K1-EJ hybrid | MKFKFNPTGTIVKKLTQYEINWYKQQYGRYPWERPVA | 4 |
| EJ-K1 hybrid | MLAKIKAMIKKFPNPYTLAAKLTTYEIAWFKNKHGYYPWEIPRC | 5 |
| GarvicinKosA | MGAIIKAGAKIVGKGVLGGGASWLGWNVGEKIWK | 6 |
| (GarA) | | |
| GarvicinKosB | MGAIIKAGAKIIGKGLLGGAAGGATYGGLKKIFG | 7 |
| (GarB) | | |
| GarvicinKosC | MGAIIKAGAKIVGKGALTGGGVWLAEKLFGGK | 8 |
| (GarC) | | |
| CereinHA | MAKIGKWVVKGAAGYLGWEIGEGIWK | 9 |
| (CerH-A) | | |
| CereinHB | MGALVKGGLKLIGGTAASWLGWEAGERVWK | 10 |
| (CerH-B) | | |
| CereinHC | MGAIIKGGLKLVGGGAAGGFTYGGLKKIFG | 11 |
| (CerH-C) | | |
| CereinHD | MGAIIKGAAKVLGKGAATGGVIYGLEKLFR | 12 |
| (CerH-D) | | |
| CereinVC | MGAVVKGGLKIIGGTAASWLGWEAGTRIWK | 13 |
| (CerV-C) | | |
| CereinVB | MGAAVKMLGKAFAGGVAGGATYGGLKKIFG | 14 |
| (CerV-B) | | |
| CereinVA | MGAVVKGALKIIGGGAASGGAVYGLERIFGR | 15 |
| (CerV-A) | | |
| CereinX A | MGKKIGKWIITGAAGWAGWEIGEGIWK | 16 |
| (CerX A) | | |
| CereinX C | MGALFKAALKAAGGGAAGGATYGGLKHFFG | 17 |
| (CerX C) | | |
| CereinX B | MKYLGTLIKGAAGGAGAYVGEKIYNWYKN | 18 |
| (CerX B) | | |
| A70A | MGKLAIKAGKIIGGGIASALGWAAGEKAVGK | 19 |
| A70B | MGAVAKFLGKAALGGAAGGATYAGLKKIFG | 20 |
| A70C | MGALIKTGAKIIGSGAAGGLGTYIGHKILGK | 21 |
| A70D | MGAVIKVGAKVIGWGAASGAGLYGLEKILKK | 22 |

### EntK1 Bacteriocin (and Its Variants Such as EntEJ97, K1-EJ and EJ-K1 Hybrids)

In a preferred aspect the second bacteriocin is a leaderless bacteriocin. EntK1 is a member of a family of leaderless bacteriocins which presently contains four members: EntK1, LsbB, EntQ and EntEJ97 (Ovchinnikov et al., 2014, J. Biol. Chem., Vol. 289, p 23838-23845). LsbB is a 30 amino acid (aa) residue peptide produced by Lactococcus lactis and it has a very narrow inhibition spectrum which contains only lactococcal strains (Gajic et al., 2003, J. Biol. Chem., Vol. 278, p 34291-34298.). The remaining three bacteriocins are produced by different enterococcal strains. EntQ (34 aa residues) and especially EntEJ97 (44 aa residues) have broader antimicrobial spectra than LsbB (Basanta et al., 2008, Int. J. Food. Microbiol., Vol. 125, p 293-307; Galvez et al., 1998, Arch. Microbiol., Vol. 171, p 59-65; Cintas et al., 2000, J. Bacteriol., Vol. 182, p 6806-6814.). EntK1 (37 aa residues) is known to inhibit L. lactis 1L1403 (Ovchinnikov et al., 2014, J. Biol. Chem., Vol. 289., p 23838-23845) and various Enterococcus species. Molecules related to EntK1 and EntEJ97 (as described in WO2018/109135, which is incorporated herein by reference) are particularly preferred second bacteriocins for use according to the invention.

Thus, in a preferred aspect, the second bacteriocin for use according to the invention is a peptide comprising an amino sequence selected from:

a)
MKFKFNPTGTIVKKLTQYEIAWFKNKHGYYPWEIPRC (EntK1),

b) a sequence with at least 40% sequence identity to sequence a),
c) a sequence consisting of at least 15 consecutive amino acids of sequence a), and
d) a sequence with at least 40% sequence identity to sequence c),
   wherein sequences b), c) and d) comprise at least the consensus sequence
   KXXXGXXPWE, wherein X may be any amino acid (SEQ ID NO:23).
   The sequence provided above is the sequence for EntK1 (SEQ ID NO:1).

In an alternative preferred aspect, in the above preferred aspect, the EntK1 sequence may instead be the EntEJ97 sequence (SEQ ID NO:2), i.e. preferably the second bacteriocin may be EntEJ97 or its related sequences as set out in b), c) or d) above (as they relate to SEQ ID NO:2).

Sequences with at least 40% sequence identity to a stated sequence are preferably at least 45, 55, 60, 65, 70, 75, 80, 85, 90, 95, 96, 97, 98 or 99% identical to that sequence (e.g. SEQ ID NO: 1 or 2 or a 15-mer portion thereof), preferably they have at least 60, 70, 80, 90 or 95% sequence identity. Such sequences comprise the consensus sequence KXXXGXXPWE. In EntK1 this sequence is KNKHGYYPWE (SEQ ID NO:24) and in EntEJ97 (SEQ ID NO:2) this sequence is KQQYGRYPWE (SEQ ID NO:25). Thus, preferably in the consensus sequence KX₁X₂X₃GX₄X₅PWE, X₁ is N or Q; X₂ is K or Q, X₃ is H or Y, X₄ is Y or R; and/or X₅ is Y (SEQ ID NO:26). Preferably this consensus sequence appears at the C-terminus of the peptide in which it appears.

As used herein “and/or” refers to one or both of the recited options being present, e.g. A and/or B includes the options i) A, ii) B or iii) A and B. A, B and/or C includes the options i) A, ii) B, iii) C, iv) A and B; v) A and C; vi) B and C, and vii) A, B and C.

Sequence identity may be determined by, e.g. using the SWISS-PROT protein sequence databank using FASTA pep-cmp with a variable pamfactor, and gap creation penalty set at 12.0 and gap extension penalty set at 4.0, and a window of 2 amino acids. Preferably said comparison is made over the full length of the sequence, but may be made over a smaller window of comparison, e.g. less than, or equal to, 50, 40, 30, 20 or 15 contiguous amino acids. Where sequences of non-identical length are compared, the comparison is over the corresponding region which shows identity, e.g. the 40% identity in d) is established by comparison to said 15 (or more) consecutive amino acids.

The second bacteriocin may comprise or consist of the above described amino acid sequences. Thus, for example, the peptide may additionally contain flanking sequences, e.g. of 1-10 amino acids at the N and/or C-terminal end. These flanking sequences may be ignored in calculating sequence identity.

The EntK1 derived peptides described herein, including the sequence identity related peptides, and peptides containing non-natural amino acids (e.g. peptidomimetics as described above) are functionally equivalent to at least one of the peptides which are set forth in the recited SEQ ID NOs 1, 2, 3 and 4 (particularly SEQ ID NOs:1 and 4). Peptides which show “functional equivalence” exhibit the same or substantially the same antibacterial effects as the peptide from which they are derived (by sequence variation or use of different amino acids). The antibacterial effects may be assessed by examining the effect of the bacteriocin on selected bacteria. Preferably a functionally equivalent peptide when tested has at least 50%, preferably at least 70, 80 or 90% of the antibacterial activity exhibited by EntK1 (and/or EntEJ97 or the described variants) on a strain of bacteria selected from the genera Enterococcus, Listeria and Lactococcus (or other bacteria on which tests have been conducted as described herein). Preferably the strain is E. faecium LMGT 3104 or E. faecalis LMGT 3358. Antibacterial activity may be determined by reference to the MIC value, minimum inhibition concentration (MIC), which is defined as the minimum amount of bacteriocin that inhibits at least 50% of the growth of the bacteria in 200 μL of culture.

Particularly preferred functionally-equivalent variants are natural biological variations (e.g. allelic variants or geographical variations). In an alternative embodiment the variants may be non-natural.

Deletions, insertions and substitutions of the disclosed peptide sequences are contemplated which provide sequences with the claimed sequence identity.

As described above, the second bacteriocin may comprise a sequence consisting of at least 15 consecutive amino acids of EntK1 (SEQ ID NO:1) or a sequence with at least 40% sequence identity to said sequence consisting of the at least 15 consecutive amino acids. In both cases, the sequence contains the above described consensus sequence. Preferably said sequence consists of at least 20, 25, 30 or 35 consecutive amino acids of EntK1, or a sequence with at least 40% sequence identity thereto. Consecutive amino acids are those which follow consecutively, without interruption, in the sequence as set forth. Preferably the consecutive amino acid sequences are chosen from the C-terminal end, e.g. the provided sequences (e.g. in SEQ ID NOs: 1 and 2) may be truncated at the N-terminal end. Preferred values for sequence identity are as described above.

Thus, in a preferred aspect the peptide may comprise an amino acid sequence selected from:
a) a sequence consisting of at least 15 consecutive amino acids of

MKFKFNPTGTIVKKLTQYEIAWFKNKHGYYPWEIPRC (EntK1)
or
MLAKIKAMIKKFPNPYTLAAKLTTYEINWYKQQYGRYPWERPVA
(EntEJ97),

wherein said at least 15 consecutive amino acids start at least 2, preferably 5 amino acids, from the N-terminal end, and
b) a sequence with at least 50% sequence identity to sequence a),
wherein sequences a) and b) comprise at least the consensus sequence
   KXXXGXXPWE, wherein X may be any amino acid.

Thus, a truncation, may be made, preferably at the N-terminal end, of 2, 3, 4, 5, 6, 7, 8, 9, 10 or up to 15 amino acids.

In a particularly preferred feature, the peptide comprises or consists of the sequence:
MIKKFPNPYTLAAKLTTYEINWYKQQYGRYPWERPVA or a sequence with at least 50% sequence identity thereto (preferably

SEQ ID NO: 3
MIKKFPNPYTLAAKLTTYEINWYKQQYGRYPWERPVA,,
EntEJ97short).

Preferred sequence identity for the above described aspects is as described hereinbefore (thus preferably, the sequence identity is at least 60, 70, 80, 90 or 95%, for example).

Furthermore, fusion (or hybrid) proteins between sequences as described herein may be made comprising at least 15 amino acids of a sequence described herein. Thus, in a further preferred aspect, the peptide comprises (or consists of) an amino acid sequence selected from:
a) a sequence consisting of at least 15 consecutive amino acids of
   MKFKFNPTGTIVKKLTQYEIAWFKNKHGYYPWEIPRC (EntK1) and at least 10, preferably 15, consecutive amino acids of

### MLAKIKAMIKKFPNPYTLAAKLTTYEINWYKQQYGRYPWERPVA (EntEJ97), and

b) a sequence with at least 50% sequence identity to sequence a),
wherein sequences a) and b) comprise at least the consensus sequence
   KXXXGXXPWE, wherein X may be any amino acid.

Thus a sequence of at least 15, 16, 17, 18, 19, 20 or up to 25 or 30 consecutive amino acids of the first (listed) sequence (SEQ ID NO:1) may be used, and conjugated or fused to a sequence of at least 10, e.g. 11, 12, 13, 14, 15 or up to 20, 25 or 30 consecutive amino acids of the second sequence (SEQ ID NO:2). The first sequence may appear at the N- or C-terminal and the second sequence at the other terminal. Preferably the first sequence appears at the N-terminal. In a preferred aspect of this embodiment, said peptide comprises or consists of the sequence:

MKFKFNPTGTIVKKLTQYEINWYKQQYGRYPWERPVA or a sequence with at least 50% sequence identity thereto (preferably
MKFKFNPTGTIVKKLTQYEINWYKQQYGRYPWERPVA, SEQ ID NO:4, K1-EJ hybrid). An alternative fusion peptide comprises or consists of the sequence provided by SEQ ID NO:5
MLAKIKAMIKKFPNPYTLAAKLTTYEIAWFKNKHGYYPWEIPRC or a sequence with at least 50% sequence identity thereto (preferably
MLAKIKAMIKKFPNPYTLAAKLTTYEIAWFKNKHGYYPWEIPRC, EJ-K1 hybrid), which also falls within the scope of the claim.

Preferred sequence identity for the above described aspects is as described hereinbefore (thus preferably, the sequence identity is at least 60, 70, 80, 90 or 95%, for example). Such variant molecules fall within the scope of embodiments described by reference to SEQ ID NO:1. In the alternative, the second bacteriocin for use in the invention is a peptide as described above without reference to SEQ ID NO:1.

In a preferred aspect, the peptide comprises or consists of:

a)
(EntK1, SEQ ID NO: 1)
MKFKFNPTGTIVKKLTQYEIAWFKNKHGYYPWEIPRC
or
(EntEJ97, SEQ ID NO: 2)
MLAKIKAMIKKFPNPYTLAAKLTTYEINWYKQQYGRYPWERPVA,

b) a sequence with at least 50%, preferably 90%, sequence identity to sequence a),
c) a sequence consisting of at least 15 consecutive amino acids of sequence a), and
d) a sequence with at least 50%, preferably 90%, sequence identity to sequence c),
wherein sequences b), c) and d) comprise at least the consensus sequence
   KXXXGXXPWE, wherein X may be any amino acid.

Preferred sequence identity for the above described aspects is as described hereinbefore (thus preferably, the sequence identity is at least 60, 70, 80, or 95%, for example). Such molecules fall within the scope of embodiments described by reference to SEQ ID NO:1. In the alternative, the second bacteriocin for use in the invention is a peptide as described above defined by reference to SEQ ID NO:2 without reference to SEQ ID NO:1.

EntK1 and EntEJ97 have 48% sequence identity across the 37 amino acids of EntK1. In this embodiment, said at least 90% sequence identity is at least 96, 97, 98 or 99% sequence identity. (Similar preferred sequence identities are applicable to other embodiments of the invention describing 90% sequence identity.) The other features of this aspect are as described hereinbefore, including the preferred aspects. In a particularly preferred aspect, the peptide comprises or consists of the sequence: MKFKFNPTGTIVKKLTQYEIAWFKNKHGYYPWEIPRC (EntK1) or

MLAKIKAMIKKFPNPYTLAAKLTTYEINWYKQQYGRYPWERPVA (EntEJ97), or a sequence consisting of at least 15 (or more, as described hereinbefore) consecutive amino acids thereof.

### GarKS Bacteriocin and its Variants

In another preferred aspect the second bacteriocin is a bacteriocin from Lactococcus garvieae, which is referred to herein as Garvicin KS, and its variants and related bacteriocins as described hereinafter. This bacteriocin is described in WO2017/084985 which is incorporated herein by reference.

Garvicin KS is a potent bacteriocin with a broad inhibition spectrum against many important problematic bacteria of genera Listeria, Staphylococcus (including S. aureus, (Chi and Holo, 2018, Curr. Microbiol., Vol. 75, p 272-277), Streptococcus and Enterococcus. Garvicin KS is capable of killing antibiotic-resistant bacteria of L. monocytogenes, MRSA and VRE which are common problematic bacteria in hospital environments.

The bacteriocin garvicin KS has three leaderless peptides with strong sequence homology. Related bacteriocins with 2-4 peptides have been identified from B. cereus (CereinH, CereinV and CereinX). A related bacteriocin from Staphylococcus is also known (Aureocin A70) with 4 peptides. The peptides from the different bacteriocins may be substituted for one another.

Thus, in a preferred aspect the second bacteriocin may be a multi-peptide complex comprising two or more peptides selected from:
a) a peptide comprising the sequence as set forth in SEQ ID NO:6 (GarA) or a sequence with at least 50% sequence identity thereto;
b) a peptide comprising the sequence as set forth in SEQ ID NO:7 (GarB) or a sequence with at least 50% sequence identity thereto; and
c) a peptide comprising the sequence as set forth in SEQ ID NO:8 (GarC) or a sequence with at least 50% sequence identity thereto;
wherein the sequence as set forth in SEQ ID NO:6 or said sequence with at least 50% sequence identity thereto comprises at least two tryptophan residues, wherein said complex has antibacterial activity.

As referred to herein a “multi-peptide complex” refers to a complex comprising at least two peptides. A complex with two peptides has been found to have antibacterial activity (see Thapa et al., 2020, Eur. J. Pharm. Sci., 151, 105333. In the above complex the two peptides are selected from the recited peptides from a), b) and c) (e.g. only two, three or four peptides). When two peptides are used they are preferably selected from a) and b) peptides. Further peptides may also be present, thus the complex may additionally comprise one or more further peptides (e.g. 4 or 5 peptides in total). As used herein “complex” refers to discrete molecules which are associated with one another through binding interactions (i.e. act as binding partners to one another) but generally do not form covalent bonds. Such molecules are “associated” with one another when they form specific interactions such that they are in contact with one another. The complex may be generated before addition to the composition or one or more of the peptides of the complex may be provided separately and form a complex in the composition.

Preferably the peptide sequences defined herein are at least 55, 60, 65, 70, 75, 80, 85, 90, 95, 96, 97, 98 or 99% identical to the sequence (SEQ ID NOs 6-8, and other peptide sequences described hereinafter, namely SEQ ID NOs 9-22) to which the peptide sequence is compared. In a preferred feature said sequence identity in any one of a), b) and/or c) is at least 60, 70, 80, 90 or 95% sequence identity.

Sequence identity may be determined as described hereinbefore.

In a preferred aspect the sequence as provided in SEQ ID NO:6 to 8 (and other peptide sequences described herein) provides the peptide to be used (i.e. has 100% sequence identity). The peptide may consist of this sequence or comprise this sequence in a longer peptide, i.e. may contain flanking sequences, e.g. of 1-10 amino acids at the N and/or C-terminal end. These flanking sequences may be ignored in calculating sequence identity. The sequences provided in the SEQ ID NOs given for the peptides may also be truncated, e.g. up to 5 amino acids from the N and/or C terminal may be removed (e.g. 1, 2, 3, 4 or 5, e.g. 1-3 amino acids at one or both terminals and/or 1-10, e.g. 1, 2, 3, 4 or 5 amino acids in total). Preferably the N-terminal amino acids are truncated. In determining the sequence identity between truncated peptides and the sequences set out in the SEQ ID NOs, the comparison window should include the truncated sequences, i.e. a truncated peptide in which 2 amino acids are removed from each end of the sequence is calculated to have a 4 amino acid mismatch and the sequence identity calculated accordingly.

Deletions, insertions and substitutions of the disclosed peptide sequences are also contemplated. In a preferred aspect, the peptide sequences in complexes of the second bacteriocin may comprise at least one, two or three deletions, insertions or substitutions relative to the sequences presented in the SEQ ID NO.

Preferably such sequence identity related peptides, and peptides containing non-natural amino acids are functionally equivalent to the peptides which are set forth in the recited SEQ ID NOs. Peptides which show “functional equivalence” exhibit the same or substantially the same antibacterial effects as the peptide from which they are derived (by sequence variation or use of different amino acids). The antibacterial effects may be assessed by providing the peptide in a complex with the other peptides which make up the bacteriocin (e.g. peptides with SEQ ID NOs: 7 and 8 in the case of a peptide derived from SEQ ID NO:6) and testing its antibacterial effects against a panel of bacteria. Preferably a functionally equivalent peptide when tested in a complex has at least 90% of the antibacterial activity exhibited by Garvicin KS on a strain of bacteria selected from the genera Enterococcus, Listeria and Staphylococcus. Preferably the strain is E. faecium LMGT 2772 or the commonly used laboratory strain L. lactis IL1403. Antibacterial activity may be determined by reference to the MIC value, minimum inhibition concentration (MIC), which is defined as the minimum amount of bacteriocin that inhibits at least 50% of the growth of the bacteria in 200 μL of culture.

Particularly preferred functionally-equivalent variants are natural biological variations (e.g. allelic variants or geographical variations). In an alternative embodiment the variants may be non-natural, or may be provided in a complex in which the different peptides are not provided together in nature.

Peptide a) as set forth above (comprising SEQ ID NO:6, GarA) or a sequence-related peptide comprises at least two tryptophan residues (or non-natural derivatized amino acids of tryptophan). These tryptophans may appear anywhere in the peptide sequence, but preferably appear in the C-terminal end of the peptide, e.g. in the 15 residues at the C-terminal end. Conveniently more than 2 tryptophans may be present, e.g. 3 or 4 tryptophans.

In a preferred aspect, at least one peptide, e.g. peptide a) comprises a consensus sequence X¹Y¹GWY²Y³GY⁴Y⁵Y⁶X²K, wherein X¹, X² and each Y may be any amino acid (SEQ ID NO:27). Preferably this consensus sequence appears at the C-terminus of the peptide in which it appears. In a particularly preferred aspect, X¹ and X² may each be any amino acid, with the proviso that at least one of X¹ and X² is a tryptophan residue. Thus the consensus sequence may be WY¹GWY²Y³GY⁴Y⁵Y⁶X²K (SEQ ID NO:28), X¹Y¹GWY²Y³GY⁴Y⁵Y⁶WK (SEQ ID NO:29) or WY¹GWY²Y³GY⁴Y⁵Y⁶WK (SEQ ID NO:30).

Particularly preferably the consensus sequence satisfies one or more of the following:
a) X¹ and X² are both tryptophan residues;
b) Y¹ is an alanine or leucine residue;
c) Y² is a glutamic acid residue;
d) Y³ is a hydrophobic amino acid residue selected from alanine, valine, leucine, isoleucine, proline, or methionine, preferably alanine, valine or isoleucine;
e) Y⁴ is a glutamic acid residue; and
f) Y⁶ is an isoleucine residue.

In a further preferred aspect the consensus sequence may have the form: Z¹Z²Z³X¹Y¹GWY²Y³GY⁴Y⁶Y⁶X²K, wherein X¹, X² and each Y and each Z may be any amino acid (SEQ ID NO:31). Preferably X¹, X² and each Y are as indicated above. In a preferred aspect Z¹ and/or Z² may be an alanine residue, and/or Z³ may be a serine or glycine residue.

Thus, for example, the consensus sequence may have the form:

(SEQ ID NO: 32)
Z¹AZ³X¹Y¹GWY²Y³GY⁴Y⁵Y⁶WK;
(SEQ ID NO: 33)
Z¹AZ³X¹Y¹GWY²Y³GY⁴Y⁵IWK;
(SEQ ID NO: 34)
AAZ³WY¹GWEY³GEY⁵IWK.

The above described family of second bacteriocins comprise family members Cerein C, Cerein X and Cerein H and these bacteriocins and related sequences form further preferred aspects for use as second bacteriocins in the invention. The peptides making up these bacteriocins are related to the peptides making up Garvicin KS as follows:

| Garvicin KS peptide | Related Cerein peptide |
|---|---|
| A: GarvicinKosA (GarA) | CereinHA (CerH-A) (SEQ ID NO: 9), |
| (SEQ ID NO: 6) | CereinHB (CerH-B) (SEQ ID NO: 10); |
| | CereinVC (CerV-C) (SEQ ID NO: 13); |
| | CereinX A (CerX A) (SEQ ID NO: 16) |
| B: GarvicinKosB (GarB) | CereinHC (CerH-C) (SEQ ID NO: 11); |
| (SEQ ID NO: 7) | CereinVB (CerV-B) (SEQ ID NO: 14); |
| | CereinX C (CerX C) (SEQ ID NO: 17) |
| C: GarvicinKosC (GarC) | CereinHD (CerH-D) (SEQ ID NO: 12); |
| (SEQ ID NO: 8) | CereinVA (CerV-A) (SEQ ID NO: 15); |
| | CereinX B (CerX B) (SEQ ID NO: 18) |

Thus, in a preferred aspect, the second bacteriocins of the invention comprise (or consist of) one or more peptides (or sequences with at least 50% sequence identity thereto or an alternative sequence identity as described hereinbefore) from each of the above groups A to C. Preferably the second bacteriocins for use in the invention contain at least one peptide containing the consensus sequence described hereinbefore.

Preferred combinations are set out below. In one aspect, preferably the peptides have the indicated sequence or in an alternative embodiment the peptides and/or the combination of peptides in the complex is non-native, i.e. does not occur in nature. The above definitions and descriptions relating to peptides of the invention apply equally to the peptides and complexes described hereinbelow.

Thus the present invention provides a composition as defined hereinbefore wherein said composition comprises a bacteriocin complex comprising two or more peptides selected from:
a) a peptide comprising the sequence as set forth in SEQ ID NO:6 (GarA) or a sequence with at least 50%, preferably 80% sequence identity thereto;
b) a peptide comprising the sequence as set forth in SEQ ID NO:7 (GarB) or a sequence with at least 50%, preferably 80% sequence identity thereto; and
c) a peptide comprising the sequence as set forth in SEQ ID NO:8 (GarC) or a sequence with at least 50%, preferably 80% sequence identity thereto;
wherein the sequence as set forth in SEQ ID NO:6 or said sequence with at least 50%, preferably 80% sequence identity thereto comprises at least two tryptophan residues. These may be considered the Garvicin KS family of bacteriocins. The at least two tryptophans are preferably in the consensus sequence described hereinbefore.

In a further preferred aspect, SEQ ID NOs: 6, 7 and 8 in the above described families of sequences may be replaced with SEQ ID NOs 9 (or 10), 11 and 12 to provide the Cerein H family of bacteriocins, SEQ ID NOs: 13, 14 and 15 to provide the Cerein V family of bacteriocins; and SEQ ID NOs: 16, 17 and 18 to provide the Cerein X family of bacteriocins. In a further aspect, the bacteriocin may be provided by Aureocin A70 which is a complex of peptides with the sequences set forth in SEQ ID NOs. 19-22, or a complex in which one or more of those peptides are replaced with a peptide having at least 50%, preferably 80% sequence identity to the peptide which it replaces.

As noted above, the different bacteriocin sub-families may be substituted for one another and thus alternative second bacteriocins are provided by a complex comprising at least two peptides selected from:
a) one or more peptide comprising a sequence as set forth in any one of SEQ ID NOs:6, 9, 10, 13 or 16 (A) or a sequence with at least 50%, preferably 80% sequence identity thereto;
b) a peptide comprising a sequence as set forth in SEQ ID NO:7, 11, 14 or 17 (B) or a sequence with at least 50%, preferably 80% sequence identity thereto; and
c) a peptide comprising a sequence as set forth in SEQ ID NO:8, 12, 15 or 18 (C) or a sequence with at least 50%, preferably 80% sequence identity thereto;
wherein the sequence as set forth in SEQ ID NO:6, 9, 10, 13 or 16 or said sequence with at least 50%, preferably 80% sequence identity thereto comprises at least two tryptophan residues and said complex has antibacterial activity.

When one or more of the a) peptides are present, preferably two or three of such peptides are present. For example, peptides comprising SEQ ID NOs: 9 and 10 (or related sequences as defined herein) may be provided in the complex. Preferred combinations are as set forth in the examples of WO2017/084985.

Preferably the peptides that are present are provided in the ratio 0.5-2:0.5-2 when two peptides are present, 0.5-2:0.5-2:0.5-2 when three peptides are present and 0.5-2:0.5-2:0.5-2:0.5-2 when four peptides are present, wherein preferably said peptides are provided in equimolar amounts in the complex or in the composition containing the peptides which will associate to form the complex.

The peptides described herein may be prepared by any convenient means known in the art, e.g. direct chemical synthesis or by recombinant means by expressing a nucleic acid molecule of the appropriate encoding sequence in a cell. Thus the peptides provided may be synthetic or recombinant, i.e. not produced in the bacteria in which they were identified. Alternatively the peptides may be produced from host cells. The peptides for use according to the invention may be isolated or purified after production, for example to a degree of purity of more than 50 or 60%, e.g. >70, 80 or 90%, preferably more than 95 or 99% purity as assessed w/w (dry weight).

The peptide(s) may be provided in the form of a composition, e.g. as described hereinafter, for the uses or methods of the invention. The composition described herein may also comprise impurities, e.g. after the preparation of said composition from one of the natural sources described herein or after synthesis of the peptides. In compositions as described herein, the peptide(s) may be present in the range of 0.001-1 mg/ml or 0.01-100 mg/ml (e.g. 0.1 to 10 mg/ml) of the composition, e.g. for use in in vitro or in vivo methods. (The provided concentrations refer to the peptide in the singular when only a single peptide is used, or the total concentration of the peptides as a complex when more than one peptide is used.)

### Antibiotics

Suitable antibiotics for use with M P1 include include penicillins (such as penicillin G, methicillin, oxacillin, cloxacillin, dicloxacillin, glucloxacillin and amoxicillin), cephalosporins (such as cephalexin (Keflex)), macrolides (such as erythromycin (E-Mycin), clarithromycin (Biaxin) and azithromycin (Zithromax)), fluoroquinolones (such as ciprofloxacin (Cipro), levofloxacin (Levaquin) and ofloxacin (Floxin)), sulfonamides (such as co-trimoxazole (Bactrim) and trimethoprim (Proloprim)), tetracyclines (such as tetracycline (Sumycin, Panmycin), minocycline and doxycycline (Vibramycin)), carbapenems (such as imipenem), aminoglycosides (such as gentamicin (Garamycin), tobramycin (Tobrex), streptomycin, amikacin and kanamycin), amphenicols (such as chloramphenicol), oxazolidinones (such as linezolid), spectinomycin, trimethoprim, rifampicin, fusidic acid, lantibiotics (such as nisin, bisin, subtilin, epidermin, gallidermin, mersacidin, actagardine, duramycin and cinnamycin) or tigecycline,.

Preferred antibiotics are erythromycin, streptomycin, tetracycline, chloramphenicol, kanamycin, rifampicin, fusidic acid, lantibiotic nisin (e.g. nisinZ) and β-lactam antibiotics. β-lactam antibiotics include penicillins (particularly pencillin G), cephalosporins, carbapenems, monobactams and β-lactamase inhibitors.

In compositions as described herein, the antibiotic, when used, may be present at a concentration in the range of 0.001-1 mg/ml or 0.01-100 mg/ml (e.g. to 10 mg/ml) in the composition, e.g. for use in in vitro or in vivo methods. This will of course depend on the particular antibiotic that is used and may be determined using methods described herein to determine the MIC when used in combination.

### Other Antibacterials

Other antibacterial agents may also be used, providing they have antibacterial activity when used alone, as described above. Such antibacterial agents include essential oil compounds such as farnesol. In a preferred feature the antibacterial agent is farnesol.

In compositions as described herein, the other antibacterial agents may be present at a concentration in the range of 0.001-1 mg/ml or 0.01-100 mg/ml (e.g. to 10 mg/ml, or 0.1 to 2 mg/ml, e.g. for farensol) in the composition, e.g. for use in in vitro or in vivo methods.

Compositions of the invention may comprise, in addition to MP1, more than one additional antibacterial agent. For example, and in preferred aspects, the compositions may comprise 2 or 3 additional antibacterial agents. Preferably in such compositions a second bacteriocin is present. In particular a combination of MP1, a second bacteriocin and another antibacterial agent (such as an antibiotic) may be used.

Preferred combinations (using two or three components as active ingredients) include:
i) MP1 and Garvicin KS (or its related molecules as described herein);
ii) MP1 and an essential oil compound such as farnesol;
iii) MP1 and a tetracycline, such as tetracycline;
iv) MP1 and an amphenicol, such as chloramphenicol;
v) MP1 and rifampicin;
vi) MP1 and fusidic acid;
vii) MP1 and an aminoglycoside, such as streptomycin or kanamycin;
viii) MP1 and a macrolide, such as erythromycin;
ix) MP1 and a lantibiotic, such as nisin;
x) MP1 and EntK1 (or its related molecules as described herein, such as K1-EJ hybrid), and optionally Garvicin KS (or its related molecules as described herein);
xi) MP1 and a β-lactam antibiotic (such as penicillin G);
xii) MP1, Garvicin KS (or its related molecules as described herein) and a β-lactam antibiotic (such as penicillin G); and
xiii) MP1, EntK1 (or its related molecules as described herein, such as EJ97short and its related molecules) and optionally a β-lactam antibiotic (such as penicillin G).

In the above combinations, Garvicin KS and its related molecules is defined by a multi-peptide complex comprising two or more peptides selected from:
a) a peptide comprising the sequence as set forth in SEQ ID NO:6 (GarA) or a sequence with at least 50% sequence identity thereto;
b) a peptide comprising the sequence as set forth in SEQ ID NO:7 (GarB) or a sequence with at least 50% sequence identity thereto; and
c) a peptide comprising the sequence as set forth in SEQ ID NO:8 (GarC) or a sequence with at least 50% sequence identity thereto;
wherein the sequence as set forth in SEQ ID NO:6 or said sequence with at least 50% sequence identity thereto comprises at least two tryptophan residues, wherein said complex has antibacterial activity.

EntK1 (and it related molecules) is defined by a peptide comprising an amino sequence selected from:

a)
MKFKFNPTGTIVKKLTQYEIAWFKNKHGYYPWEIPRC (EntK1),

b) a sequence with at least 40% sequence identity to sequence a),
c) a sequence consisting of at least 15 consecutive amino acids of sequence a), and
d) a sequence with at least 40% sequence identity to sequence c),
wherein sequences b), c) and d) comprise at least the consensus sequence
   KXXXGXXPWE, wherein X may be any amino acid (SEQ ID NO:23). K1-EJ hybrid is defined by a peptide which comprises or consists of the sequence:
MKFKFNPTGTIVKKLTQYEINWYKQQYGRYPWERPVA or a sequence with at least 50%, preferably 80% sequence identity thereto (preferably
   MKFKFNPTGTIVKKLTQYEINWYKQQYGRYPWERPVA, SEQ ID NO:4, K1-EJ hybrid). EJ97short and its related molecules is defined by a peptide which comprises or consists of the sequence: MIKKFPNPYTLAAKLTTYEINWYKQQYGRYPWERPVA or a sequence with at least 50%, preferably 80% sequence identity thereto (preferably
   MIKKFPNPYTLAAKLTTYEINWYKQQYGRYPWERPVA, SEQ ID NO:3, EntEJ97short). The preferred embodiments of these families are as described hereinbefore.

The composition has antibacterial activity against a broad spectrum of bacteria. Preferably the composition has antibacterial activity against at least one bacteria selected from the genera Bacillus, Streptococcus, Listeria, Enterococcus, Staphylococcus, Acinetobacter and Paenibacillus. Especially preferably the composition has antibacterial activity against at least one bacteria selected from the species Bacillus cereus, Listeria monocytogenes, Listeria innocua, Listeria grayi, Listeria seelingeri, Streptococcus thermophylus, Streptococcus agalactia, Streptococcus pneumonia, Streptococcus salivarius, Enterococcus faecalis, Enterococcus faecium, Staphylococcus aureus, Staphylococcus hemolyticus, Staphylococcus pseudintermedius, Acinetobacter nosocomialis and Paenibacillus larvae, particularly preferably Methicillin-resistant Staphylococcus aureus (MRSA), Methicillin-resistant Staphylococcus pseudintermedius (MRSP), Vancomycin-resistant Enterococci (VRE) and antibiotic-resistant strains of Listeria monocytogenes. Preferably said composition has antibacterial activity against at least one bacteria from each of the genera Bacillus, Streptococcus, Listeria, Enterococcus, Staphylococcus, Acinetobacter and Paenibacillus. Preferred strains against which the compositions have activity are provided in the Examples. In a preferred aspect the compositions have activity against one or more bacterial strain resistant to antibacterial agents, e.g. resistant to methicillin, pencillin and/or vancomycin, e.g. methicillin and/or vancomycin-resistant strains, e.g. as described herein.

As described hereinafter, the compositions have particular utility in various therapeutic and prophylactic methods and uses. To achieve the methods and uses of the invention the active ingredients, i.e. MP1 and the additional antibacterial agent(s) may be appropriately modified for use in a pharmaceutical or a composition for use in preparing food products or other items or products. For example the composition may be stabilized against degradation for example by the use of appropriate additives such as salts or non-electrolytes, acetate, SDS, EDTA, citrate or acetate buffers, mannitol, glycine, HSA or polysorbate.

In a preferred aspect the antibacterial agents and MP1 are solubilized in hydroxypropyl cellulose (HPC), particularly for topical treatments (e.g. a 1-10% w/v, e.g. 5% w/v solution). HPC is a synthesized cellulose-derivative to which a hydroxypropyl group is introduced as the substitute of 2,3,6-OH group, thereby rendering it soluble in both water and organic solvents.

Thus, the compositions may be provided in the form of a pharmaceutical composition comprising in addition one or more pharmaceutically acceptable diluents, carriers or excipients, which composition forms a further aspect of the invention and may be for use in therapy as described herein. Similar diluents, carriers or excipients may also be provided in compositions for non-pharmaceutical compositions but are not necessarily of pharmaceutical grade, e.g. for antibacterial protection of products. “Pharmaceutically acceptable” as referred to herein refers to ingredients that are compatible with other ingredients of the compositions as well as physiologically acceptable to the recipient.

The compositions described herein may be formulated in a conventional manner with one or more physiologically acceptable (where necessary) carriers, excipients and/or diluents, according to techniques well known in the art using readily available ingredients. Thus, the active ingredients may be incorporated, optionally together with other active substances as a combined preparation, with one or more conventional carriers, diluents and/or excipients, to produce conventional galenic preparations such as tablets, pills, powders (for topical administration or inhalation), lozenges, sachets, cachets, elixirs, suspensions (as injection or infusion fluids), emulsions, solutions, syrups, sprays, aerosols (as a solid or in a liquid medium), ointments, soft and hard gelatin capsules, suppositories, sterile injectable solutions, sterile packaged powders, and the like. Biodegradable polymers (such as polyesters, polyanhydrides, polylactic acid, or polyglycolic acid) may also be used for solid implants. The compositions may be stabilized by use of freeze-drying, undercooling or Permazyme.

Suitable excipients, carriers or diluents are lactose, dextrose, sucrose, sorbitol, mannitol, starches, gum acacia, calcium phosphate, calcium carbonate, calcium lactose, corn starch, aglinates, tragacanth, gelatin, calcium silicate, microcrystalline cellulose, polyvinylpyrrolidone, cellulose, water syrup, water, water/ethanol, water/glycol, water/polyethylene, glycol, propylene glycol, methyl cellulose, methyl hydroxybenzoates, propyl hydroxybenzoates, talc, magnesium stearate, mineral oil or fatty substances such as hard fat or suitable mixtures thereof. Agents for obtaining sustained release formulations, such as carboxypolymethylene, carboxymethyl cellulose, cellulose acetate phthalate, or polyvinylacetate may also be used.

The compositions may additionally include lubricating agents, wetting agents, emulsifying agents, viscosity increasing agents, granulating agents, disintegrating agents, binding agents, osmotic active agents, suspending agents, preserving agents, sweetening agents, flavouring agents, adsorption enhancers (e.g. surface penetrating agents or for nasal delivery, e.g. bile salts, lecithins, surfactants, fatty acids, chelators), browning agents, organic solvent, antioxidant, stabilizing agents, emollients, silicone, alpha-hydroxy acid, demulcent, anti-foaming agent, moisturizing agent, vitamin, fragrance, ionic or non-ionic thickeners, surfactants, filler, ionic or non-ionic thickener, sequestrant, polymer, propellant, alkalinizing or acidifying agent, opacifier, colouring agents and fatty compounds and the like.

The compositions of the invention may be formulated so as to provide quick, sustained or delayed release of the active ingredients after administration to the body by employing techniques well known in the art.

The composition may be in any appropriate dosage form to allow delivery or for targeting particular cells or tissues, e.g. as an emulsion or in liposomes, niosomes, microspheres, nanoparticles or the like with which the active ingredients may be absorbed, adsorbed, incorporated or bound. This can effectively convert the product to an insoluble form. These particulate forms may overcome both stability (e.g. degradation) and delivery problems.

These particles may carry appropriate surface molecules to improve circulation time (e.g. serum components, surfactants, polyoxamine908, PEG etc.).

The use of solutions, sprays, suspensions, gels and emulsions are preferred, e.g. the active ingredients may be carried in water, a gas, a water-based liquid, an oil, a gel, an emulsion, an oil-in water or water-in-oil emulsion, a dispersion or a mixture thereof.

Compositions may be for topical (i.e. to the skin or mucosal membranes), oral or parenteral administration, e.g. by injection. Injections may be used to provide systemic effects or to provide local effects at the site of infection e.g. intramammary injection for mastitis.

Topical compositions and administration are however preferred, and include gels, creams, ointments, sprays, lotions, liniments, salves, sticks, soaps, powders, films, aerosols, drops, foams, solutions, emulsions, suspensions, dispersions e.g. non-ionic vesicle dispersions, milks and any other conventional pharmaceutical or cosmetic forms in the art.

Ointments, gels and creams may, for example, be formulated with an aqueous or oily base with the addition of suitable thickening and/or gelling agents. Lotions may be formulated with an aqueous or oily base and will, in general, also contain one or more emulsifying, dispersing, suspending, thickening or colouring agents. Powders may be formed with the aid of any suitable powder base. Drops and solutions may be formulated with an aqueous or non-aqueous base also comprising one or more dispersing, solubilising or suspending agents. Aerosol sprays are conveniently delivered from pressurised packs, with the use of a suitable propellant.

Alternatively, the compositions may be provided in a form adapted for oral or parenteral administration. Alternative pharmaceutical forms thus include plain or coated tablets, capsules, suspensions and solutions containing the active component optionally together with one or more inert conventional carriers and/or diluents, e.g. with corn starch, lactose, sucrose, microcrystalline cellulose, magnesium stearate, polyvinylpyrrolidone, citric acid, tartaric acid, water, water/ethanol, water/glycerol, water/sorbitol, water/polyethylene glycol, propylene glycol, stearyl alcohol, carboxymethylcellulose or fatty substances such as hard fat or suitable mixtures thereof.

In an alternative to providing the active ingredients in a composition, they may instead be provided in separate solutions or compositions allowing different mechanisms or timings for administration or application. As referred to herein “co-administration” and “co-application” refers to use of all of the active ingredients in the same method rather than simultaneous use (either in terms of timing or in the same composition). The composition (or MP1 and at least one additional antibacterial agent) may be used in vitro, ex vivo or in vivo as described herein.

In view of the antibacterial properties of the compositions of the invention they may be used for therapeutic or prophylactic purposes. Thus, the present invention provides a composition as defined hereinbefore (or MP1 and at least one additional antibacterial agent as described hereinbefore) for therapy. In particular the compositions (or MP1 and at least one additional antibacterial agent) may be used in treating or preventing bacterial infection. The compositions (or MP1 and at least one additional antibacterial agent) may be suitable for treating humans or for veterinary use.

Thus, in a further aspect the present invention provides a composition as defined hereinbefore (or MP1 and at least one additional antibacterial agent as defined hereinbefore) for use in treating or preventing a bacterial infection in a subject or patient. Alternatively the invention provides use of a composition as defined hereinbefore (or MP1 and at least one additional antibacterial agent as defined hereinbefore) in the preparation of a medicament for treating or preventing a bacterial infection in a subject or patient. Alternatively described the invention provides a method of treating or preventing a bacterial infection comprising administering a composition as defined hereinbefore (or MP1 and at least one additional antibacterial agent as defined hereinbefore) to a patient or subject or a part of said subject's or patient's body. Furthermore, the present invention provides a product containing the components of the composition of the invention (i.e. MP1 and at least one additional antibacterial agent as defined hereinbefore) and optionally one or more additional active ingredients as a combined preparation for simultaneous, separate or sequential use in human or animal therapy, preferably as described herein. Also provided is a kit separately comprising each of the active ingredients of the composition of the invention, preferably for a use or method as described herein.

Preferably said kit (or product) is for simultaneous, separate or sequential use in a medical treatment or for prophylaxis as described herein.

As defined herein “treatment” (or treating) refers to reducing, alleviating or eliminating one or more symptoms of the bacterial infection which is being treated, relative to the symptoms prior to treatment. Such symptoms may be correlated with the abundance of bacteria present on the treated patient or subject. “Prevention” (or preventing or prophylaxis) refers to delaying or preventing the onset of the symptoms of the bacterial infection. Prevention may be absolute (such that no bacterial infection occurs) or may be effective only in some individuals or for a limited amount of time.

As referred to herein a “bacterial infection” is invasion of bodily tissue by a bacteria that proliferates at that site and which may result in injury to that tissue. Preferably the bacterial infection is a skin infection (preferably caused by Staphylococcus, e.g. by Staphylococcus aureus, Staphylococcus hemolyticus or Staphylococcus pseudintermedius or by Enterococcus, e.g. by Enterococcus faecium or Enterococcus faecalis), an oral or throat infection (preferably caused by Streptococcus), an infection present in or causing dental caries (preferably caused by Streptococcus) or mastitis (preferably caused by Staphylococcus or Streptococcus).

In a particularly preferred aspect, the present invention provides an antibacterial composition as defined herein for use in treating or preventing mastitis in a milk-producing subject or patient, wherein preferably said subject or patient is a mammalian animal, preferably a human, cow, sheep, horse, pig or goat. As referred to herein a milk-producing animal refers to a lactating female mammalian animal, preferably a livestock animal.

Particularly preferred are bacterial infections in skin and soft tissue infections (SSTIs). In a particularly preferred aspect the skin infection is present in a wound. Preferably the bacteria is in the form of a biofilm. Preferably the bacteria is a gram-positive bacteria.

Preferably the bacterial infection is caused by (and the composition is used to treat or prevent the bacterial infection from) at least one bacteria selected from the genera Bacillus, Streptococcus, Listeria, Enterococcus, Staphylococcus, Acinetobacter and Paenibacillus, preferably selected from the species Bacillus cereus, Listeria monocytogenes, Listeria innocua, Listeria grayi, Listeria seelingeri, Streptococcus thermophylus, Streptococcus agalactia, Streptococcus pneumonia, Streptococcus salivarius, Enterococcus faecalis, Enterococcus faecium, Staphylococcus aureus, Staphylococcus hemolyticus, Staphylococcus pseudintermedius, Acinetobacter nosocomialis and Paenibacillus larvae. In a particularly preferred aspect the bacterial infection is caused by at least one bacterial strain selected from Methicillin-resistant Staphylococcus aureus (MRSA), Methicillin-resistant Staphylococcus pseudintermedius (MRSP), Vancomycin-resistant Enterococci (VRE) and antibiotic-resistant strains of Listeria monocytogenes. As described herein MRSP includes fucidin-resistant strains. In particularly preferred treatments or preventions, the bacterial strain is one or more bacterial strain disclosed in the Examples herein. In a further preferred aspect, the bacterial infection is caused by one or more bacterial strain resistant to antibacterial agents, e.g. resistant to methicillin, pencillin and/or vancomycin.

In in vitro or ex vivo uses, bacteria may be present (although an infection may not be present where the location of the bacteria is not a bodily tissue) on the item or object to be treated. This may be in the form of a biofilm.

In a preferred aspect the compositions described herein (or MP1 and at least one additional antibacterial agent as defined hereinbefore) may used to treat or prevent a bacterial infection as follows:

Preferred combinations (using two or three components as active ingredients) include:
i) MP1 and Garvicin KS (or its related molecules as described herein) for treating a bacterial infection caused by S. aureus and/or MRSA and/or S. hemolyticus;
ii) MP1 and an essential oil compound such as farnesol for treating a bacterial infection caused by S. aureus and/or MRSA;
iii) MP1 and a tetracycline, such as tetracycline, for treating a bacterial infection caused by S. aureus and/or MRSA and/or S. pseudointermedius and/or MRSP;
iv) MP1 and an amphenicol, such as chloramphenicol, for treating a bacterial infection caused by S. aureus and/or MRSA and/or S. pseudointermedius and/or MRSP;
v) MP1 and rifampicin for treating a bacterial infection caused by S. aureus and/or MRSA;
vi) MP1 and fusidic acid for treating a bacterial infection caused by S. aureus and/or MRSA and/or S. pseudointermedius and/or MRSP;
vii) MP1 and an aminoglycoside, preferably streptomycin or kanamycin for treating a bacterial infection caused by S. pseudointermedius and/or MRSP;
viii) MP1 and a macrolide, preferably erythromycin for treating a bacterial infection caused by S. pseudointermedius and/or MRSP;
ix) MP1 and a lantibiotic, preferably nisin for treating a bacterial infection caused by S. aureus and/or MRSA;
x) MP1 and EntK1 (or its related molecules as described herein, such as K1-EJ hybrid) for treating a bacterial infection caused by E. faecium, E. faecalis and/or S. hemolyticus;
xi) MP1, EntK1 (or its related molecules as described herein, such as K1-EJ hybrid) and Garvicin KS (or its related molecules as described herein) for treating a bacterial infection caused by S. hemolyticus;
xii) MP1 and a β-lactam antibiotic (such as penicillin G) for treating a bacterial infection caused by S. aureus and/or MRSA;
xiii) MP1, Garvicin KS (or its related molecules as described herein) and a β-lactam antibiotic (such as penicillin G) for treating a bacterial infection caused by S. aureus and/or MRSA and/or E. faecalis and/or S. pseudintermedius; and
xiv) MP1, EntK1 (or its related molecules as described herein, such as EJ97short and its related molecules) and optionally a β-lactam antibiotic (such as penicillin G) for treating a bacterial infection caused by S. pseudintermedius and/or MRSP.

Animals (or patients/subjects) to which the compositions (or MP1 and at least one additional antibacterial agent as defined hereinbefore) may be applied or administered include mammals, reptiles, birds, insects and fish particularly during fish aquaculture (e.g. salmon or cod). Preferably the animals to which the compositions of the invention (or MP1 and at least one additional antibacterial agent) are applied are mammals, particularly primates, domestic animals, livestock and laboratory animals. Thus preferred animals include mice, rats, rabbits, guinea pigs, cats, dogs, monkeys, pigs, cows, goats, sheep and horses. Especially preferably the compositions (or MP1 and at least one additional antibacterial agent) are applied, or administered, to humans. A “part” of the subject or patient refers to a body part or area to be treated, e.g. an infected region of the skin or other organ of the body.

The administration may be by any suitable method known in the medicinal arts, including for example oral, parenteral (e.g. intramuscular, subcutaneous, intraperitoneal or intravenous), intestinal, percutaneous, buccal, rectal or topical administration or administration by inhalation. The preferred administration forms will be administered orally (e.g. in food for animals), or most preferably topically. As will be appreciated oral administration has its limitations if the active ingredients are digestible. To overcome such problems, ingredients may be stabilized as mentioned previously. In a particularly preferred aspect the bacterial infection is an infection on the skin and/or the composition (or its components) is administered topically.

It will be appreciated that since the active ingredients for performance of the invention takes a variety of forms, e.g. peptides, the form of the composition and route of delivery will vary. Preferably, however, liquid solutions, creams or suspensions would be employed, particularly e.g. for oral delivery or topical administration.

The concentration of active ingredients in compositions of the invention (or when used separately), depends upon the nature of the compound used (i.e. the particular antibacterial agents that are used), the mode of administration, the course of treatment, the age and weight of the patient/subject, the medical indication, the body or body area to be treated and may be varied or adjusted according to choice. (The timing of the treatment is affected by similar factors.) Generally however, appropriate concentration ranges for the various components of the composition are as described hereinbefore, i.e. in a concentration range of 0.001-1 mg/ml for MP1, the antibiotic and any other antibacterial agent, when used (with the preferred ranges as indicated previously). The total of the active ingredients of the composition may comprise 0.0001, 0.0005, 0.001 or 0.01 to 25%, e.g. 0.0005-15%, e.g. 0.01 to 10%, such as 0.1 or 0.5 to 5, e.g. 1-5% (w/w) of the final preparation for administration, determined based on the total weight of all the components, particularly for topical administration. Effective single doses for compositions of the invention (or components thereof) may lie in the range of from 0.01-10mg/cm/day, preferably 0.1-1 mg/cm/day, when applied topically, depending on the animal being treated, taken as a single dose (again taking into account the total weight of the components used).

A single dose of the composition of the invention may be used, but alternatively, repeated doses may be used, e.g. the dose may be repeated two or more times, e.g. three, four or five times, as necessary. The interval for repeated doses may be every 12 hours to 7 days, e.g. once every 24 to 48 hours, e.g. daily. In a preferred aspect, where necessary, 2-5 doses are used with an interval of 24-48 hours between doses.

In a further aspect, the present invention provides use of a composition as defined hereinbefore (or MP1 and at least one additional antibacterial agent as defined hereinbefore) as an antibacterial for in vitro, in vivo or ex vivo methods. This use may be on an item or object. Preferably the antibacterial is effective against bacterial infection from a bacteria as described hereinbefore. The composition (or its components) may also be used to prevent the development of antibiotic resistance in a bacteria by applying or administering the composition (or its components) to the bacteria. Again this may be on an item or object.

It will be appreciated from the comments herein and the results presented in the Examples that MP1 acts to improve the activity of other antibacterial agents when used in combination with those antibacterial agents. This allows lower amounts of the various antibacterial agents to be used. Furthermore, MP1 may be used to re-sensitize bacteria to antibacterial agents to which they have become resistant thus allowing previously ineffective agents to be used. Thus in a further aspect the present invention provides use of MP1 as an adjuvant in an antibacterial composition. As used herein, an “adjuvant” is an entity which serves to improve or increase the activity of a co-administered entity, e.g. to sensitize the bacteria to the co-administered entity, e.g. when administered in vitro or in vivo.

The antibacterial compositions (or MP1 and at least one additional antibacterial agent) may be used to preserve food products to prevent their spoilage. Thus, in a further aspect, the present invention provides a method of preparing a preserved food product comprising adding a composition as defined hereinbefore (or MP1 and at least one additional antibacterial agent as defined hereinbefore) to a food product. A “preserved” food product refers to a food product to which a composition of the invention (or MP1 and at least one additional antibacterial agent as defined hereinbefore) has been applied to provide antibacterial (preservative) properties. A “food product” is an edible product that may be consumed by animals which provides nutritional benefits. Food products include in particular animal-derived food products, such as dairy and meat products as well as plant-derived food products. Various foods and beverages which may be susceptible to bacterial infection are contemplated.

The invention thus further provides a preserved food product comprising a food product and a composition as defined hereinbefore (or MP1 and at least one additional antibacterial agent as defined hereinbefore).

The invention also provides a method of avoiding food spoilage comprising mixing a food product with a composition as defined hereinbefore (or MP1 and at least one additional antibacterial agent as defined hereinbefore). Food “spoilage” refers to a reduction in the nutritional properties, decay or bacterial infection of food. The food is mixed with the composition (or its components) in appropriate proportions to provide beneficial antibacterial properties but without substantial deleterious effects on the taste or nutritional properties of the food product. Appropriate concentrations may be readily determined by methods known in the art.

The compositions of the invention (or MP1 and at least one additional antibacterial agent as defined hereinbefore) may also be used to provide antibacterial properties to non-food items, e.g. medical products. Thus, in a further aspect the present invention provides an item covered, impregnated, or coated with a composition as defined hereinbefore (or MP1 and at least one additional antibacterial agent as defined hereinbefore). The invention also provides a method of disinfecting or decontaminating an item, of bacteria present on said item, comprising covering, impregnating, or coating said item with a composition of the invention (or components, i.e. active ingredients, thereof).

The “item” refers to any inanimate object. Preferably the item is a medical device, instrument, implement or equipment, a prosthetic or material, tissue or wound dressing. Medical devices include pacemakers and heart valves, medical implements include catheters and scalpels, medical equipment includes gloves and other clothing, prosthetics or material include artificial joints, breast implants and scaffold material. Wound dressings include plasters and bandages as well as cements, glues or matrices which may be used for wound repair.

The invention also provides a personal health care product (including cosmetic products) comprising a composition as defined hereinbefore (or MP1 and at least one additional antibacterial agent as defined hereinbefore). The product may be a product which is susceptible to bacterial contamination or which may be used to provide antibacterial protection to the body to which it is applied. Thus, the health care products may be body, face or lip milks, foams, sprays, lotions, creams, gels or balms, make-up products (such as eye or face products, including eye shadow, powder, lipstick, foundation, mascara, blush, eyeliner, nail polish, tinted creams and foundations, sun make-up), creams, lotions or colourants, hair products such as hair rinse, spray mist, gel, mousse, shampoo, conditioner, lotion, emulsion or colouring product and oral health or dental products such as toothpaste, mouthwash, mouth gel or spray, lozenge or chewing gum. Preferably the product is toothpaste, mouthwash, skin cream, lotion or spray.

The item or product is covered, impregnated, coated or mixed with the composition (or its components) in appropriate proportions to provide beneficial antibacterial properties but without substantial deleterious effects on the item or product, e.g. its functional properties. Appropriate concentrations and methods of covering, impregnation or coating may be readily determined by methods known in the art.

A method of preparing the above described item or health care product comprising applying a composition of the invention (or MP1 and at least one additional antibacterial agent as defined hereinbefore) to said item or product, or mixing or impregnating said item or product with said composition (or its components), forms a further aspect of the invention. The use of a composition of the invention (or its components) to prepare such items or products is also considered an object of the invention.

The present invention also provides in vitro methods of killing, damaging or preventing the replication of bacteria comprising administering a composition as defined hereinbefore (or MP1 and at least one additional antibacterial agent as defined hereinbefore) to said bacteria. The bacteria may be present on an object or item. Relevant definitions for killing, damaging and preventing replication as provided hereinbefore are also relevant to this aspect of the invention. Preferred aspects of the bacteria to be killed (etc.) and the composition and its components are as set out hereinbefore.

The methods described in the Examples form further preferred aspects of the invention. All combinations of the preferred features described above are contemplated, particularly as described in the Examples. The invention will now be described by way of non-limiting Examples with reference to the drawings in which:

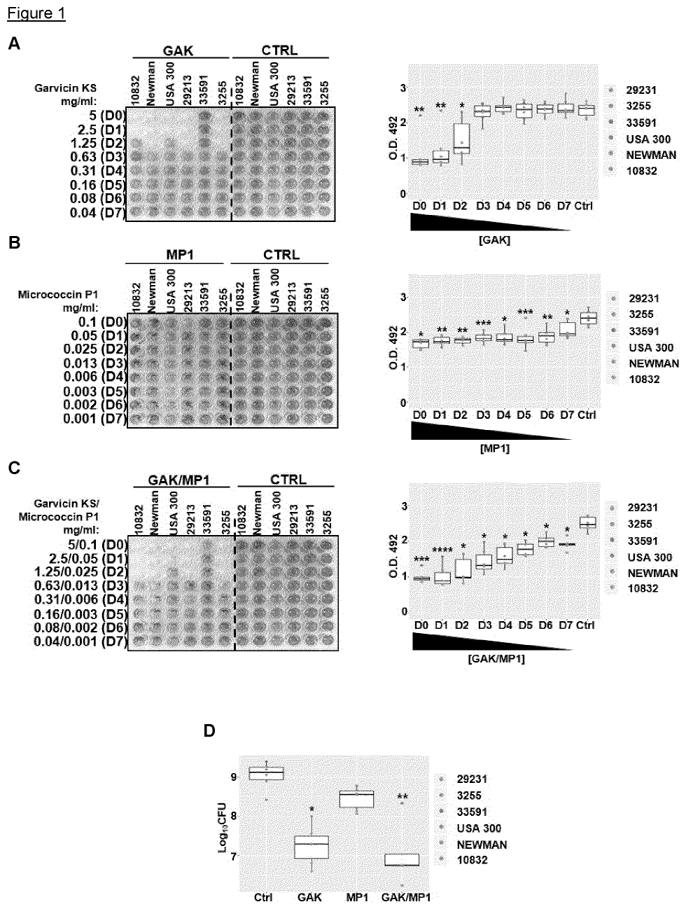

FIG. 1 shows the assessment of Garvicin KS and micrococcin P1 as individuals (A and B) or in combination (C) in eradicating S. aureus biofilms. The left panels show representative images of the BOAT assay performed with serial two-fold dilutions of the antimicrobials (first six columns of the plate) for the indicated strains. The concentration (in mg/ml) of the antimicrobials in the dilutions (dilution factors: D0 to D7) is indicated on the far left of the images. At the same time, the assay was also performed using the control vehicles to their final concentrations (Ctrl, the last six columns of each plate): 0.02% (v/v) TFA for garvicin KS, 0.013% (v/v) trifluoracetic acid/6.25% (v/v) 2-propanol for micrococcin P1 and the mixture 0.033% (v/v) trifluoracetic acid/6.25% (v/v) 2-propanol for the combination. The development of red colour indicates the retention of metabolic activity, and its quantification was performed by optical density readings at 492 nm (O.D. 492). The boxplots in the right panel show the trends of recovery of the bacterial metabolic activity as a function of the dilution factor of the different antimicrobials. Each box displays the median distribution (thick line within boxes) and the degree of variability (amplitude of the box and whiskers) of the metabolic activities for the indicated strains measured at increasing dilution factors (from D0 to D7). (D), boxplot showing the median distribution of logarithmic colony formation unit (LogCFU) values for the indicated strains. The LogCFU counting was performed following the BOAT assay for each indicated antimicrobial. The concentrations used were 5 mg/ml for garvicin KS and 0.1 mg/ml for micrococcin P1. The control (Ctrl) samples were treated with an equivalent amount of the antimicrobial vehicles. The data represent the average values obtained from three independent experiments.

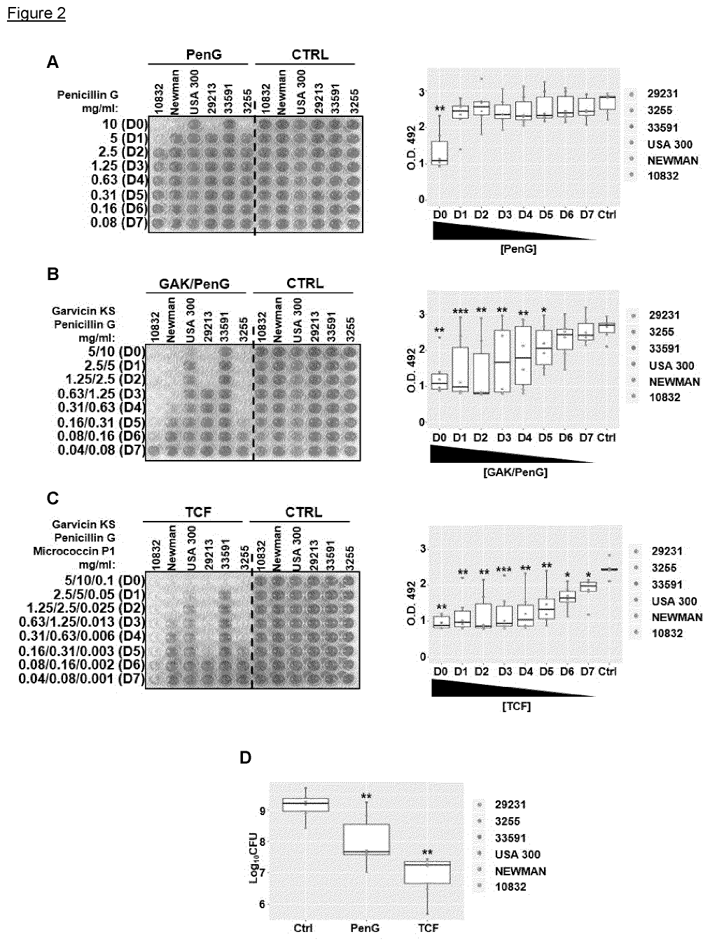

FIG. 2 shows that Garvicin KS and micrococcin P1 sensitize MRSA strains to penicillin G. A), a representative image of the BOAT assay performed with penicillin G (Pen G, first six columns of the plate) or with its control vehicle (Ctrl, last six columns of the plate) is shown. The relative boxplot analyzing the trends of metabolic activity as a function of penicillin G dilution is shown on the far right. The BOAT assay and the relative metabolic activity quantifications were performed as detailed in FIG. 1. In B) and C), similar experiments as in FIG. 2A, but the data refer to the combination treatment between garvicin KS and penicillin G (GAK/PenG), and the tricomponent formulation (TCF) between garvicin KS, micrococcin P1 and penicillin G, respectively. The control vehicles to their final concentrations were sterile distilled water in panel A, 0.02% (v/v) TFA in panel B and 0.033% (v/v) trifluoracetic acid/6.25% (v/v) 2-propanol in panel C. D), boxplot showing the median distribution of LogCFU values obtained upon the indicated treatments and strains. The LogCFU counting was performed following the BOAT assay for each indicated antimicrobial. The concentrations used for garvicin KS and micrococcin P1 were the same as in FIG. 1D, and the penicillin G concentration was 10 mg/ml. The control (Ctrl) samples were treated with an equivalent amount of the antimicrobial vehicles. All the data represent the average values obtained from three independent experiments.

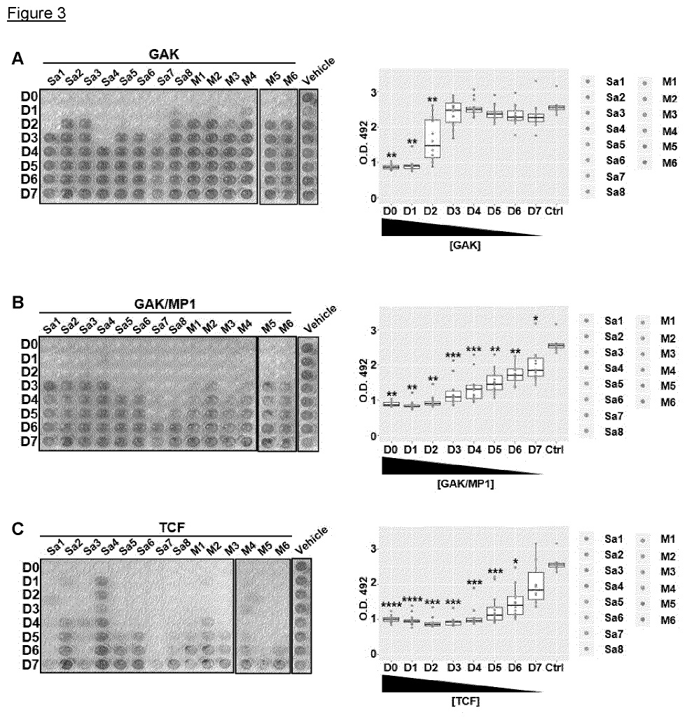

FIG. 3 shows the sensitivity of the clinical S. aureus strains to garvicin KS alone (A), in combination with micrococcin P1 (B), and the tricomponent formulation (C). Representative images of the BOAT assay performed are shown in the left panel while the corresponding boxplots analyzing the trend of metabolic activity recovery are shown in the right panel. The assays were performed on 8 methicillin-sensitive (Sa1-8) and on 6 methicillin-resistant (M1-6) strains. In the vehicle columns, the strain MRSA6 (M6) shown on the right side was used as a representative of the outcome for all vehicle-treated strains. The BOAT assays, antimicrobial vehicles and the relative quantifications were performed as indicated in FIGS. 1 and 2. All the data represent the average values obtained from three independent experiments. For the control vehicles of the antimicrobials see the legends in FIGS. 1 and 2.

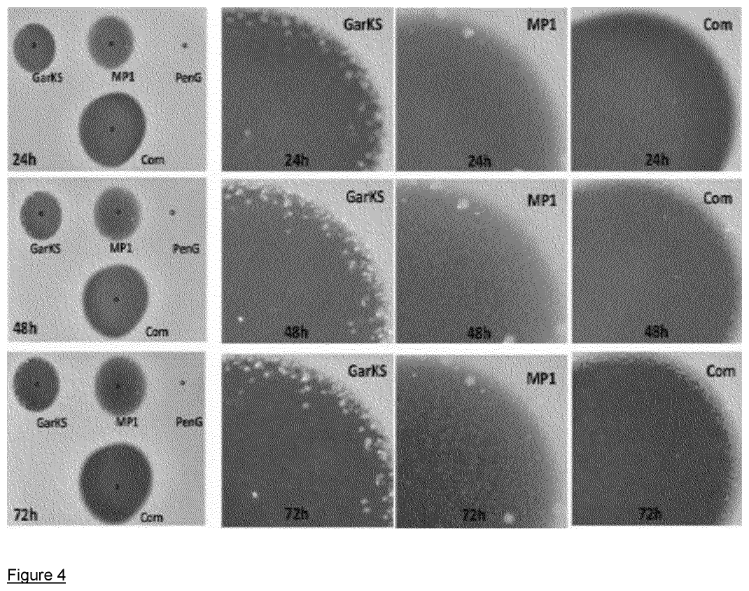

FIG. 4 shows the assessment of the antimicrobials—GarKS, MP1 and PenG, individually and in combination (Com) against MRSA ATCC 33521-lux. Pure GarKS and PenG each at 50 μg, MP1 at 0.1 μg, were applied at the indicated spots. The pictures of the plates were taken after 24, 48 and 72 h of incubation at 37° C. Sections of the inhibition zones on the right show resistance development seen as small white dots (in the inhibition zones of GarKS and MP1).

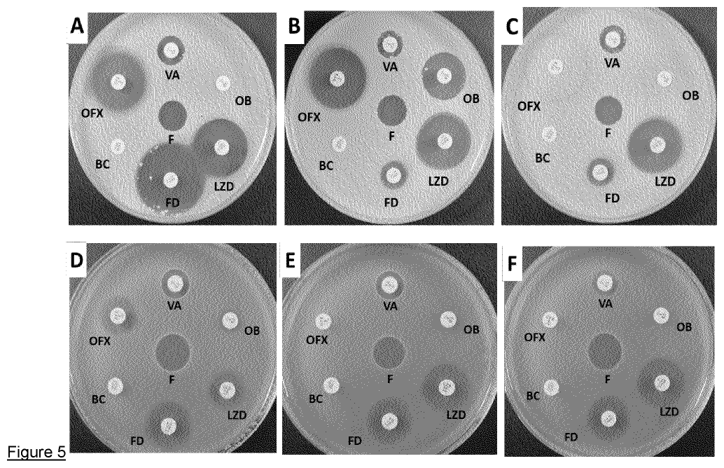

FIG. 5 shows a comparison of antimicrobial activity of the Formulation with selected antibiotics against S. pseudintermedius (A-C) and Enterococcus faecalis (D-F). Formulation (F) containing 15 μg for GarKS, 15 μg for PenG and 0.3 μg for MP1; Antibiotic discs—VA-vancomycin, 5 μg; OB—cloxacillin, 5 μg; LZD—linezolid, 10 μg; FD—fusidic acid, 10 μg; BC—bacitracin, 0.04 Units; OFX—ofloxacin 5 μg.

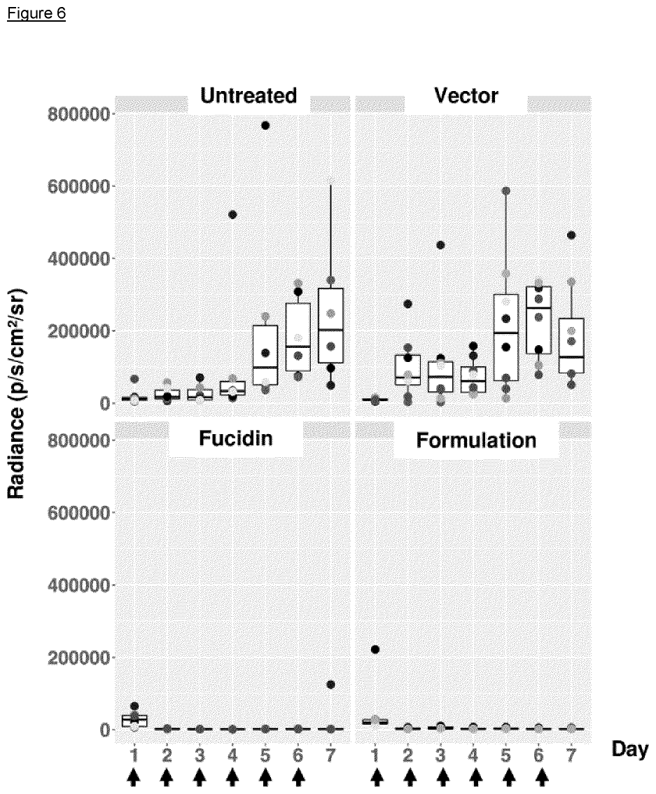

FIG. 6 shows boxplots of bioluminescent signal produced by MRSA ATCC33591-lux in photons/second/cm²/steradian in differently treated mouse groups. The days of treatment with Formulation, Fucidin cream and Vector are indicated with arrows. Untreated mice were only infected and left untreated. The area within each box represents the interquartile region (IQR), which comprises the second and third quartile and describes the interval of values where the middle 50% of the observed data are distributed. The thick black line within each box represents the median value. The extension of the IQR (box height) expresses the degree of variability measured within the middle 50% of the observed data, with “whiskers” extending out at either side of the boxes marking the minimum and maximum observed values as well as the variability outside the middle 50% of values (whisker length). Outliers are displayed as data that extend out of the whisker limit (1.5xIQR). The number of mice was 6 in the untreated group, 8 in the Vector group; 8 in the Fucidin group and 7 in the Formulation group.

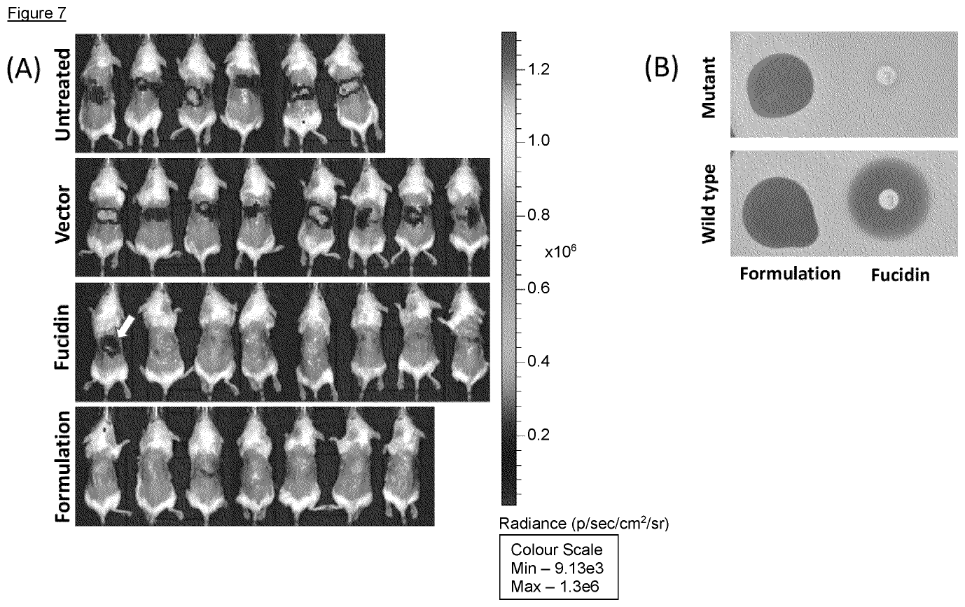

FIG. 7 shows (A) In vivo imaging of bioluminescent signal produced by MRSA ATCC33591-lux in photons/second/cm²/steradian from the different mouse groups on the last day of the experiment (7 days post infection, after six treatments). The white arrow in the Fucidin group indicates a Fucidin-resistant mutant population appearing on the mouse after 6 consecutive days of treatments (see FIG. 6). (B) Fucidin resistance develops during the treatment of mice. Bacterial cells isolated from the wound with strong bioluminescent signal in the Fucidin treated group (white arrow in A) were rechallenged and showed to be resistant to Fusidic acid disc but not to the Formulation. Wildtype MRSA ATCC33591-lux cells exposed to the formulation and Fucidin were sensitive to both antimicrobials.

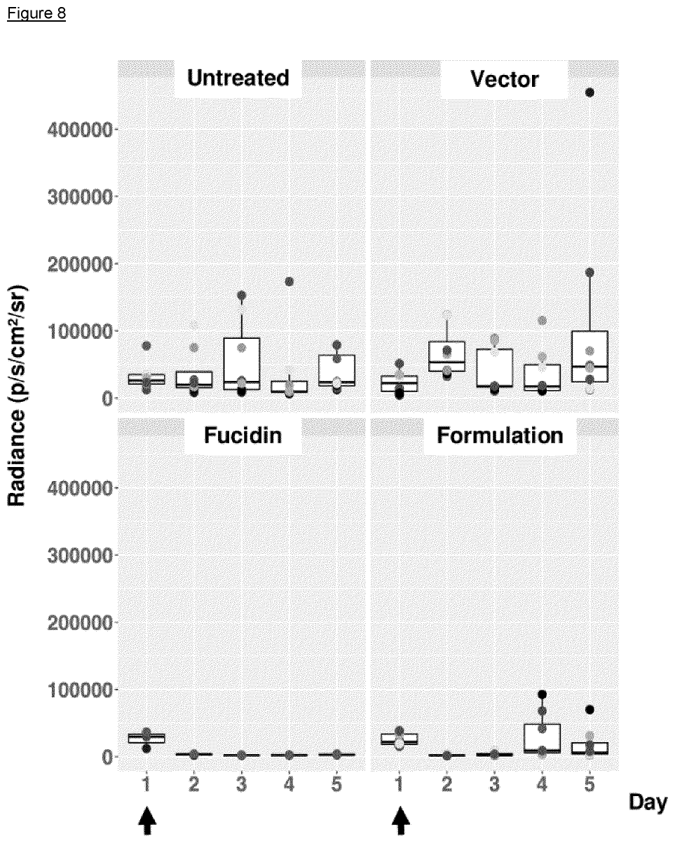

FIG. 8 shows boxplots of bioluminescent signal produced by MRSA ATCC33591-lux in photons/second/cm²/steradian in differently treated mouse groups. Application of Formulation, Fucidin cream and Vector (5% HPC) was done only once on day 1 post infection (arrow). Untreated mice were only infected and left untreated. The boxplots description is the same as in FIG. 6. The number of mice was 8 in all groups except for the Fucidin group which was 4.

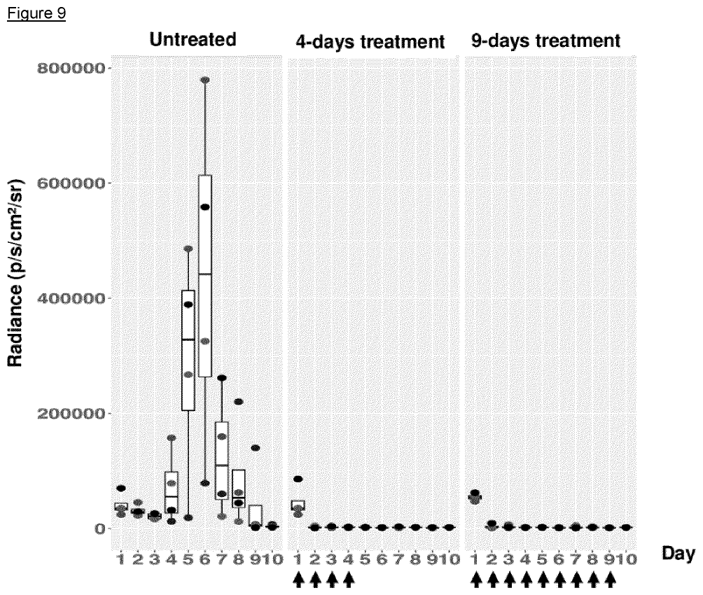

FIG. 9 shows boxplots of bioluminescent signal produced by MRSA ATCC33591-lux in photons/second/cm²/steradian in differently treated mouse groups. Application of the Formulation was done only four times (4-days treatment) or nine times (9-days treatment). The days of treatment are indicated by arrows. The untreated group was infected but not treated. The boxplots description is the same as in FIG. 6. The number of mice was 4 in each group.

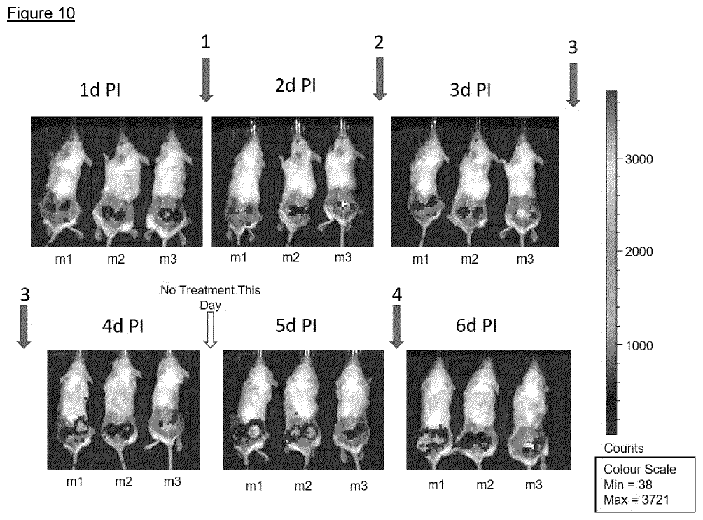

FIG. 10 shows in vivo imaging of bioluminescent signal in photons/second/cm²/steradian from the different mouse groups on each day of the treatment with tetracycline (M1), MP1 (M2) and tetracycline and MP1 (M3). The arrows show the days of treatment.

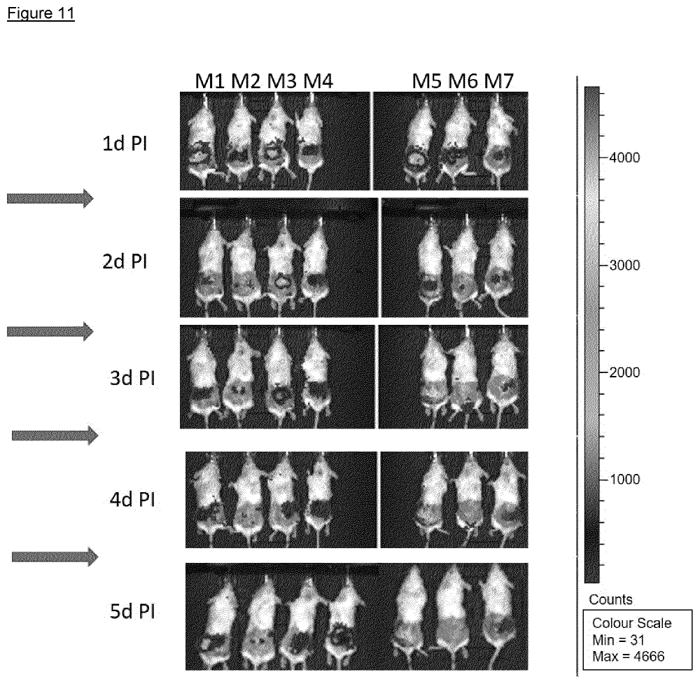

FIG. 11 shows in vivo imaging of bioluminescent signal in photons/second/cm²/steradian from the different mouse groups on each day of the treatment with rifampicin (M1, M2), MP1 (M3, M4), rifampicin+MP1 (M5, M6) or no treatment (M7). The arrows show the days of treatment.

### EXAMPLE 1

### Effect of Micrococcin 1 With Garvicin KS and Penicillin on Methicillin-Resistant Staphylococcus aureus in Liquid or Biofilms

### Materials and Methods

### Bacterial Strains and Growth Conditions.

Staphylococcus aureus strains were grown O/N in tryptic soy broth (TSB) (Sigma) at 37° C. in aerobic conditions without shaking. The clinical S. aureus strains were obtained from Blue Peter Public Health and Research Centre (BPHRC), LEPRA Society, Hyderabad, India and were isolated from plantar ulcers of patients suffering from leprosy (see below).

### Bacterial Strain Isolation From Infected Leprosy-Associated Skin Ulcers

Leprosy patients with plantar ulcers who were registered at LEPRA Society, BPHRC, Hyderabad, India during December 2018 through May 2019 (n=31) were enrolled into the study after obtaining an informed consent. The study protocol was approved by the Institutional Ethical Committee at LEPRA Society. A total of thirty-one wound swabs were collected using sterile pure viscose swab (Hi-media, Mumbai, India) and transported immediately for further processing to the microbiology laboratory. One swab was collected from each leprosy foot ulcer. Each swab was inoculated on Blood and McConkey agar and incubated at 37° C. overnight and the plates were examined for growth on the following day. Cultures were identified by colony morphology, Gram staining and other standard tests. In addition, all the isolates were subjected to species level identification by MALDI-TOF MS (matrix-assisted laser desorption/ionization time of flight mass spectrometry) (Vitek MS system, bioMerieux, France) as per the manufacturer's instructions. The identity of the bacterial species was further confirmed by 16S rRNA genotyping. All wound swabs yielded mono-bacterial culture. Out of 31 isolates, the majority of the species identified, 45.16% (14/31), were confirmed to be S. aureus isolates and all were biofilm producers. Additional bacterial species were S. haemolyticus, Corynebacterium striatum, Proteus spp., Moraxella spp., Enterococcus faecalis, Pseudomonas spp., Citrobacter koseri.

### Antimicrobials and Vehicles

Garvicin KS peptides, (GAK-A, GAK-B and GAK-C, or GarA, GarB and GarC) were synthesized by Pepmic Co., LTD, China with 90-99% purity. Micrococcin P1 was purchased from Cayman Chemical, Michigan, USA with A5% purity. Garvicin KS was solubilized to concentrations of 10-30 mg/ml in 0.1% trifluoracetic acid (TFA) (Sigma). Micrococcin P1 was solubilized in a 50% (v/v) mixture of isopropanol (Merck) with 0.1% (vol/vol) TFA. Penicillin G (Sigma) was solubilized to a stock concentration of 100 mg/ml. Antibiotic discs were from Oxoid. All antimicrobials and antibiotics were stored at −20° C. until use.

### Planktonic Cell Growth Inhibition Assays and Antimicrobial Synergy Determination

Growth inhibition assays were performed in 96-well microtiter plates as described previously (Chi and Holo, 2018, supra). Briefly, 135 μl of TSB were dispensed in each well of a microtiter plate (according to the number of bacterial strains tested) except in the wells of the first row. The antimicrobials were diluted in TSB to working concentrations in a final volume of 285 μl and dispensed in the wells of the first row. The working concentrations were 100 μg/μl for garvicin KS, 10 μg/μl for micrococcin P1 and 1 mg/ml for penicillin G. From the first row, 150 μl of the antimicrobials were then serially diluted (two-fold dilutions) in a sequential fashion until the last row of the plate. Finally, 15 μl of a fresh O/N culture of each strain were added in the appropriate wells to reach a final volume of 150 μl in each well. The plates were then incubated at 37° C. for 24 h. The growth inhibition was expressed as minimum inhibitory concentration (MIC₅₀), which refers to the minimum concentration of the antimicrobial needed to reduce at least 50% of the microbial growth compared to the untreated control. The MIC₅₀ was assessed by optical density readings at 600 nm (O.D.₆₀₀).

Synergistic interactions between antimicrobials were determined using the fractional inhibition concentration (FIC). The FIC values were calculated as follows: the sum of the FIC =FICa+FICb+FICc, where the FICa means MIC of A in combination/MIC of A alone, FICb means MIC of B in combination/MIC of B, and FICc means MIC of C in combination/MIC of C alone. Effects were considered as synergistic if FIC was ≤0.5 for two components mixture and ≤0.75 for three components mixture.

### Kirby-Bauer Disc Diffusion Susceptibility Test

The efficacy of the antibiotics was assessed using the Kirby-Bauer disc diffusion susceptibility protocol (Hudzicki, 2009, ASM Microbe Library, Disk Diffusion Susceptibility Test Protocol). One day prior to the inoculum preparation, the microorganisms were subcultured. Using a sterile inoculating loop, five well-defined colonies were touched and suspended in 5 mL of sterile BHI broth and incubated until a cell density equal to 0.5 McFarland standard (˜1×10⁸ CFU/mL) was achieved. The inoculum was then spread on the surface of Mueller-Hinton agar (MHA) plates. Agar plates were left to dry in a sterile hood for 10-15 minutes before antibiotic discs (Oxoid) were applied with a disc dispenser (Oxoid). The inhibition zones were evaluated after a 24 h incubation at 37° C.

### In Vitro Biofilm Production and Biofilm Formation Ability Assay

Staphylococcus aureus strains were inoculated in 5 ml of TSB and incubated O/N at 37° C. Ten μl of the O/N cultures were then inoculated in 90 μl of TSB supplemented with 1% glucose and 1% NaCl (TSB-GN) in the appropriate wells of a 96-well microtiter plate (Sarstedt) to a final volume of 100 μl. The plates were then incubated at 37° C. for 24 h. After the incubation, the presence of the biofilm at the bottom of the wells was assessed visually.

Biofilm formation ability assays were performed as described previously (Stepanovic et al., 2007, APMIS, Vol. 115, p 891-899) with some modifications. Staphylococcus aureus biofilms were allowed to form for 24 h prior to being washed twice with 100 μl of 0.9% NaCl saline buffer at room temperature (RT) to remove the planktonic cells and left to dry for 15 minutes. After drying, 200 μl of a 0.4% solution of crystal violet (Sigma) were added to each well and incubated for an additional 15 minutes. The dye was then removed and the wells were washed three times with 200 μl of 0.9% NaCl saline buffer, and the biofilm-bound crystal violet was then extracted by incubating the wells with 100 μl 70% ethanol. The extraction procedure was repeated twice and the combined crystal violet amount extracted was quantified by O.D. reading at 600 nm. The quantification of the crystal violet released from the biofilm is a surrogate measure of the amount of bacterial cells forming the biofilm. Blank controls consisted of 150 μl of plain TSB-GN.

### Biofilm-Oriented Antimicrobial Test (BOAT)

The BOAT assay was performed as described previously (Gronseth et al., 2017, Int. J. Pediatr. Otorhinolaryngol., Vol. 103, p 58-64) with some modifications. The serial dilutions of the antimicrobials were prepared in challenge plates as follows: 175 μl of TSB were transferred in each row of a 96-well microtiter plate, except for the first row, according to the number of microbial strains to treat. In the first row of the plate, the antimicrobials were diluted to their respective working concentrations in a final volume of 350 μl of TSB. From the first row, 175 μl of the antimicrobial dilutions were then transferred to the second row of the plate and further serially diluted to the bottom of the plate. The same procedure was followed to prepare the controls, except that instead of the antimicrobials an equivalent volume of the respective vehicles were used. The biofilms were allowed to form for 24 h and then washed twice with 100 μl of sterile saline buffer and a total of 150 μl of the antimicrobial and control dilutions were transferred from the challenge plate to the corresponding wells of the biofilm plate. The challenged biofilms were then incubated for an additional 24 h at 37° C. After the challenge period, the antimicrobial dilutions were removed and the biofilms were carefully washed three times with 150 μl of sterile saline buffer. A total of 100 μl of TSB supplemented with 0.025% of triphenyl-tetrazolium chloride (TTC, Sigma) were then added to each well of the plate and further incubated at 37° C. for 5h. The results were then assessed by monitoring the development (or not) of red formazan (red colour), denoting the retention of metabolic activity by bacterial cells (Moussa et al., 2013, J. Mycol., Vol. 2013; Perez et al., 2010, Lett. Appl. Microbiol., Vol. 51, p 331-337). The medium was then removed and 200 μl of an ethanol:acetone (70:30) mixture was added to the wells and incubated O/N in order to extract the red formazan. The amount of extracted dye, reflecting the degree of bacterial cell metabolic activity, was then quantified by spectrophotometric readings at 492 nm.

### Determination of the Bacterial Viability After BOAT

The procedure for the BOAT assay was repeated as described above except that instead of adding the TTC solution, the antimicrobial-challenged cells were resuspended in TSB and then serially diluted in TSB buffer. Serial dilutions of the bacterial cells were than plated on BHI agar plates and incubated at 37° C. for 24 h. The results were then assessed by direct counting of the developed colonies and the colony forming unit (CFU) was determined.

### Laser Scanning Confocal Microscopy

Biofilms were allowed to form for 24 h as described above with the exception that they were formed in the wells of chambered cover-glass plates (Thermo Fisher Scientific) before being challenged with the antimicrobials or the respective control vehicles diluted in TSB. Biofilms were then treated with the LIVE/DEAD Biofilm Viability Kit (Molecular Probes, Thermo Fisher Scientific) according to the manufacturer's instructions. Z-stacks of the stained biofilms were then taken on a confocal laser scanning microscope (Zeiss), using a 488 nm argon laser line for exciting the SYTO-9 (green) dye and a 561 nm laser line for the propidium iodide (red).

### Scanning Electron Microscopy (SEM)

For this analysis S. aureus biofilms were grown on 8 mm rounded glass coverslips for 24 h before being challenged with the antimicrobials or the respective control vehicles diluted in TSB. After the antimicrobial treatment, the biofilms were carefully washed twice in phosphate buffered saline (PBS) and then fixed in 3% glutaraldehyde O/N. Subsequently, biofilms were subjected to dehydration in increasing alcohol series of 30, 50, 70, 90, 96% ethanol for 10 minutes each, followed by 4×10 minutes in 100% ethanol. The samples were then subjected to critical point drying and sputter-coated with a palladium-gold thin film before examination by SEM system (Zeiss) at 15 kV.

### Statistical Analysis

All quantifications are representative of three independent experiments. All the data analysis and graphical representations were performed with R Studio (Version 1.0.15).

### Results

The In Vitro Ability of Garvicin KS to Eradicate S. aureus Biofilms is Strain-Dependent and Improved Antimicrobial Activity Was Observed Using Garvicin KS in Combination With Micrococcin P1

The antimicrobial potential of the bacteriocins micrococcin P1 and garvicin KS has never been assessed on clinically relevant S. aureus strains in a biofilm setting.

In order to start exploring the antimicrobial activity of MP1 and garvicin KS, we tested their antimicrobial activity against six S. aureus strains available in our collection, that displayed a good biofilm forming activity (data not shown), and a variegated susceptibility to common antibiotics, with three strains (USA 300, ATTC 29213 and ATTC 33591) resistant to penicillin (data not shown). For planktonic cells garvicin KS and micrococcin P1 displayed MIC₅₀ values ranging from 25 to 50 μg/ml and 0.6 to 10 μg/ml, respectively (Table 1). It is interesting to note that the two bacteriocins synergized against the two MRSA strains, USA300 and ATTC 33591, among the six tested. It has been suggested that upon formation of a biofilm, pathogens can become 10-1000 times less susceptible to antimicrobials (Ito et al., 2009, Appl. Environ. Microbiol., Vol. 75, p 4093-4100, Mah and O'Toole, 2001, Trends Microbiol., Vol. 9, p 34-39), we therefore used the MIC data produced for planktonic cells as a basis to set higher working concentrations to test on biofilms. These were 5 mg/mI for garvicin KS and 0.1 mg/ml for micrococcin P1.

In order to test whether these two bacteriocins retained their antimicrobial effects on S. aureus biofilms, we used a modified version of the biofilm-oriented antimicrobial test (BOAT), where the metabolic activity indicator triphenyl-tetrazolium chloride was used to assess the susceptibility of the bacterial strains to the antimicrobials tested (Gronseth et al., 2017, Int. J. Pediatr. Otorhinolaryngol., Vol. 103, p 58-64; Moussa et al., 2013, J. Mycol., Vol. 2013). Biofilms from the selected S. aureus strains were allowed to form for 24 h before BOAT assays were performed using the bacteriocins indicated in FIG. 1 or their respective control (Ctrl) vehicles (see Figure legend). As can be observed in FIG. 1, garvicin KS alone was sufficient to eradicate the biofilm-associated metabolic activity produced by five out of six S. aureus strains, including the MRSA strains USA 300, with MIC values ranging between 1.3 and 2.5 mg/ml (FIG. 1A, and Table 2). Conversely, the S. aureus strain ATCC 33591 remained insensitive to the treatment, highlighting an over 100-fold MIC increase compared to the planktonic state (Table 2). Similarly, and in contrast with the results obtained in liquid culture, micrococcin P1 was ineffective against all the strains at a concentration of 0.1 mg/ml (FIG. 1B and Table 2), indicating that some of the biofilm producers became over 160-fold more resistant to micrococcin P1 compared to the planktonic state. Interestingly, while the combination of the two bacteriocins failed to promote a synergistic effect for most strains, except for ATTC 10832 and 3255 (Table 2), it indeed brought about a steady reduction of the metabolic activity for all the tested strains (FIG. 1C).

In order to monitor more directly the cell viability after the antimicrobial treatment, we performed a BOAT assay followed by CFU counting. The treatment with garvicin KS and micrococcin P1 was repeated as above, but instead of adding the metabolic activity indicator, the remaining cells in the wells were serially diluted and spot-plated on agar dishes followed by incubation at 37° C. and CFU counting; the results of this assay are shown in FIG. 1D. As can be seen, and in line with the metabolic activity profiles, the treatments with garvicin KS and the combination Garvicin KS/micrococcin P1 led to the most dramatic reduction in LogCFU levels for all the strains. Consistent with result of the BOAT assays S. aureus ATTC 33591 was the strain that retained the highest viability among those tested, representing the upper outlier for the combination treatment (FIG. 1D). In addition, it is interesting to note that although the metabolic activity of most of the strains was apparently abolished when garvicin KS was used, alone or in combination with micrococcin P1 at high concentrations (5 mg/ml), the CFU counting evidenced that variable levels of cell viability were indeed retained.

Taken together, these data confirm previous studies that biofilms indeed form a protective environment for bacteria leading to a dramatic increase of the resistance to antimicrobials and indicate that garvicin KS, alone or in combination with micrococcin P1, represents a potential antimicrobial compound alternative to antibiotics for the treatment of biofilm-related S. aureus infections.

The Combined Treatment Garvicin KS and Micrococcin P1 Sensitizes MRSA S. aureus Strains Against Penicillin G

BOAT assays were performed to assess the degree of sensitivity of S. aureus biofilms to a β-lactamic antibiotic, penicillin G, and to assess the effects of the combination of the antibiotic with garvicin KS and micrococcin P1. The maximum concentration of penicillin G was set to 10 mg/ml considering the MIC values obtained for planktonic cells (Table 3). In line with their antibiotic sensitivity profiles (data not shown), the treatment with penicillin G failed to inhibit the growth of two MRSA strains (USA300 and ATTC 33591) (FIG. 2A). The antibiotic abolished the metabolic activity of biofilms produced by the other strains although only at the highest concentration tested (FIG. 2A and Table 4). This further confirms that biofilm formation strongly diminishes the sensitivity of bacteria to antibiotics. On the other hand, the combination of the antibiotic with garvicin KS produced a strong reduction in the MIC values of the non-MRSA strains (FIG. 2B and Table 4) but, it failed to inhibit the growth of the two MRSA strains, USA300 and ATTC 33591. Strikingly, however, the further addition of micrococcin P1 in a tricomponent formulation (TCF) inhibited also the metabolic activity of the MRSA strains (FIG. 2C and Table 4).

In line with these data, the BOAT assay followed by CFU counting confirmed that the penicillin G treatment led to a reduction in the viability of the non-MRSA strains, and the further addition of garvicin KS and micrococcin P1 reduced more substantially also the viability of the MRSA strains (FIG. 2D). Furthermore, it is interesting to note that although the LogCFU median values obtained upon the treatment with three component formulation (TCF) was higher when compared with the garvicin KS/micrococcin P1 combination (FIG. 1D), albeit not significantly (p=0.2123; paired Student's t-test); the treatment with the tricomponent formulation led to a strong and statistically significant reduction (p=0.0106; paired Student's t-test) of the viability of the MRSA strain ATTC 33591 (FIG. 2D), which was the most poorly affected strain by any other treatment.

We were next interested in performing a more in-depth visual investigation of the biofilms produced in vitro and of the effects elicited by the treatments described above within the biofilms. In order to address this point, we performed a LIVE/DEAD biofilm staining followed by confocal microscopy analysis by using a combination of two fluorophores: SYTO-9 and propidium iodide (PI), which were previously shown to selectively stain live and dead bacterial cells, respectively (Gronseth et al., 2017, supra; Drago et al., 2016, J. Chemother., Vol. 28, p 383-389). The strains ATTC 10832 and ATTC 33591 were chosen for this analysis as representatives of a methicillin-sensitive and -resistant strains, respectively. The control vehicle (Ctrl)-treated samples produced biofilms with a thickness up to 12 μm and largely dominated by SYTO-9-positive (green—live) bacterial cells for both tested strains (data not shown). In good agreement with the results presented above, the treatment with the antimicrobials described in FIGS. 1 and 2, produced strain-dependent effects. For ATTC 10832, with the exception of micrococcin P1, all the treatments promoted a dramatic shift in the staining pattern; with a strong increase in the proportion of PI-positive (red—dead) cells (data not shown). The ATTC 33591 strain, on the other hand, showed an increased resistance towards the treatment with garvicin KS and penicillin G, whereas the combined treatments with garvicin KS/micrococcin P1 (GAK/M P1) and particularly with the tricomponent formulation (TCF), strongly impacted its pattern of cell viability (data not shown).

Taken together, these results indicate that the combined treatment with garvicin KS and micrococcin P1 sensitizes S. aureus MRSA strains to β-lactam antibiotics. Furthermore, these results also demonstrate that the use of a combinatory formulation between bacteriocins and antibiotics produce a strong growth-inhibitory and synergistic effect against S. aureus strains characterized by an increased resistance to antimicrobials.

The Formulation and Its Components Are Effective in Abolishing the Growth of S. aureus Clinical Strains

Having found that garvicin KS, either alone or in combination with micrococcin P1 and penicillin G, is effective in eradicating the biofilms produced from the S. aureus strains tested so far, we moved on to assessing the antimicrobial effects of these combinations on S. aureus strains derived from nosocomial skin infections. Towards this end, we obtained a panel of bacterial strains isolated from infected skin ulcers derived from patients affected by leprosy in India. A total of 31 strains were obtained from the Blue Peter Research Centre, of which 14 were confirmed to be S. aureus isolates, and all displayed a good biofilm formation ability in vitro (data not shown). When we repeated the BOAT assay on biofilms produced with the clinical isolates, garvicin KS alone showed antimicrobial activity against all the strains (FIG. 3A) albeit at low dilution factors, with a pattern of recovery of the metabolic activity that resembled that obtained in FIG. 1A (FIG. 3A, right panel). In good agreement with the data presented above, the addition of micrococcin P1 and penicillin G further increased the efficacy of the formulation and progressively extended the range of dilution factors that retained antimicrobial activity (FIG. 3B and C).

### The Tricomponent Formulation Causes Visible Cell Damage in Treated Staphylococcal Biofilms

To further characterize the effect of the tricomponent formulation, S. aureus biofilms were allowed to develop on the surface of glass slides, and then treated for 24 h with the tricomponent formulation and the vehicle as control. The morphology of biofilm architecture was then analyzed using scanning electron microscopy (SEM). Consistent with previous studies, the control-treated biofilm appeared as large and multilayered aggregates of cells with poor or interspersed extracellular polymeric substance (EPS) surrounding the cell clusters (Chin et al., 2015, BMC Genomics, Vol. 16, p 471; Kong et al., 2018, Sci. Rep., Vol. 8, p 2758; Wu et al., 2019, J. Orthop. Surg. Res., Vol. 14, p 10). Conversely, at low magnifications the formulation-treated biofilm appeared to have a lower cell density (and a reduced thickness) compared to the control, as larger areas of the underlying glass surface could be observed (data not shown). At higher magnifications it became apparent that cells undergone severe damage as their morphology appeared irregular and deformed compared to control-treated cells (data not shown). In addition, the formulation treatment also produced a significant amount of larger particles on the surface of the cells, which we speculate to be cell contents or debris from damaged cells.

TABLE 1
MIC₅₀ values for garvicin KS and micrococcin P1 determined in liquid culture for
the indicated strains. Antimicrobial concentrations are expressed in mg/ml.

| | Strain | | | | | |
|---|---|---|---|---|---|---|
| | ATTC | | USA | ATTC | ATTC | S.a. |
| Antimicrobial | 10832 | Newman | 300 | 29213 | 33591 | 3255 |
| Individual | | | | | | |
| component | | | | | | |
| Garvicin KS | 2.5 × 10⁻² | 2.5 × 10⁻² | 2.5 × 10⁻² | 2.5 × 10⁻² | 5 × 10⁻² | 2.5 × 10⁻² |
| Micrococcin P1 | 6.3 × 10⁻⁴ | 3.13 × 10⁻⁴ | 6.3 × 10⁻⁴ | 2.5 × 10⁻³ | >1 × 10⁻² | 1 × 10⁻² |
| Combination | | | | | | |
| Garvicin KS | 6.3 × 10⁻³ | 3.13 × 10⁻³ | 1.56 × 10⁻³ | 1.3 × 10⁻² | 1.3 × 10⁻² | 1.3 × 10⁻² |
| Micrococcin P1 | 6.3 × 10⁻⁴ | 3.13 × 10⁻⁴ | 1.56 × 10⁻⁴ | 1.3 × 10⁻³ | 1.3 × 10⁻³ | 1.3 × 10⁻³ |
| FIC* | 1.25 | 1.13 | 0.31 | 1.04 | 0.39 | 0.65 |

*Synergy is achieved when fractional inhibition concentration (FIC) ≤ 0.5.

TABLE 2
MIC₅₀ values for garvicin KS and micrococcin P1 determined in biofilms for
the indicated strains. Antimicrobial concentrations are expressed in mg/ml.

| | Strain | | | | | |
|---|---|---|---|---|---|---|
| | ATTC | | USA | ATTC | ATTC | S.a. |
| Antimicrobial | 10832 | Newman | 300 | 29213 | 33591 | 3255 |
| Individual | | | | | | |
| components | | | | | | |
| Garvicin KS | 2.5 | 1.25 | 2.5 | 1.25 | >5 | 1.25 |
| Micrococcin | >1.0 × 10⁻¹ | >1.0 × 10⁻¹ | >1.0 × 10⁻¹ | >1.0 × 10⁻¹ | >1.0 × 10⁻¹ | >1.0 × 10⁻¹ |
| P1 | | | | | | |
| Combination | | | | | | |
| Garvicin KS | 1.25 | 1.25 | 5 | 1.25 | >5 | 3.1 × 10^{-1.} |
| Micrococcin | 2.5 × 10⁻² | 2.5 × 10⁻² | 1.0 × 10⁻¹ | 2.5 × 10⁻² | >1.0 × 10⁻¹ | 2.5 × 10^{-2.} |
| P1 | | | | | | |
| FIC* | 0.75 | 1.25 | 1.5 | 1.25 | ND | 0.5 |

*Synergy is achieved when fractional inhibition concentration (FIC) ≤ 0.5.
ND: not determined. The MIC values exceeded the maximum concentration tested.

TABLE 3
MIC₅₀ values for Penicillin G, garvicin KS and micrococcin P1 determined in liquid culture
for the indicated strains. Antimicrobial concentrations are expressed in mg/ml.

| | Strain | | | | | |
|---|---|---|---|---|---|---|
| | ATTC | | USA | ATTC | ATTC | S.a. |
| Antimicrobial | 10832 | Newman | 300 | 29213 | 33591 | 3255 |
| Individual | | | | | | |
| components | | | | | | |
| Penicillin G | <7.8 × 10⁻³ | <7.8 × 10⁻³ | >1.0 | 1.3 × 10⁻¹ | >1.0 | <7.8 × 10⁻³ |
| Combination | | | | | | |
| Garvicin KS | <7.8 × 10⁻⁴ | <7.8 × 10⁻⁴ | 2.5 × 10⁻² | 1.3 × 10⁻² | 5 × 10⁻² | <7.8 × 10⁻⁴ |
| Penicillin G | <7.8 × 10⁻³ | <7.8 × 10⁻³ | 2.5 × 10⁻¹ | 1.3 × 10⁻¹ | 5 × 10⁻¹ | <7.8 × 10⁻³ |
| FIC* | ND | ND | 1.25 | 1.52 | 1.5 | ND |
| Combination | | | | | | |
| Garvicin KS | <7.8 × 10⁻⁴ | <7.8 × 10⁻⁴ | 6.3 × 10⁻³ | 3.1 × 10⁻³ | 1.3 × 10⁻² | <7.8 × 10^{-4.} |
| Micrococcin | <7.8 × 10⁻⁵ | <7.8 × 10⁻⁵ | 6.3 × 10⁻⁴ | 3.1 × 10⁻⁴ | 1.3 × 10⁻³ | <7.8 × 10⁻⁵ |
| P1 | | | | | | |
| Penicillin G | <7.8 × 10⁻³ | <7.8 × 10⁻³ | 6.3 × 10⁻² | 3.1 × 10⁻² | 1.3 × 10⁻¹ | <7.8 × 10⁻³ |
| FIC** | ND | ND | 1.32 | 0.49 | 0.52 | ND |

*Synergy with fractional inhibition concentration (FIC) ≤ 0.5.
**Synergy with fractional inhibition concentration (FIC) ≤ 0.75.
ND: not determined. The MIC values exceed the minimum concentration tested.

TABLE 4
MIC₅₀ values for penicillin G. garvicin KS and micrococcin P1 determined in biofilms
for the indicated strains. Antimicrobial concentrations are expressed in mg/ml.

| | Strain | | | | | |
|---|---|---|---|---|---|---|
| | ATTC | | USA | ATTC | ATTC | S.a. |
| Antimicrobial | 10832 | Newman | 300 | 29213 | 33591 | 3255 |
| Individual | | | | | | |
| components | | | | | | |
| Penicillin G | 10 | 10 | >10 | 10 | >10 | 10 |
| Combination | | | | | | |
| Garvicin KS | 3.1 × 10⁻¹ | 6.3 × 10⁻¹ | >5 | 1.25 | >5 | 3.1 × 10⁻¹ |
| Penicillin G | 6.3 × 10⁻¹ | 1.3 | >10 | 2.5 | >10 | 6.3 × 10⁻¹ |
| FIC* | 0.12 | 0.63 | ND | 1.25 | ND | 0.31 |
| Combination | | | | | | |
| Garvicin KS | 1.6 × 10⁻¹ | 1.25 | 5 | 3.1 × 10⁻¹ | 5 | 3.1 × 10⁻¹ |
| Micrococcin P1 | 3.1 × 10⁻³ | 2.5 × 10⁻² | 1 × 10⁻¹ | 6.3 × 10⁻³ | 1 × 10⁻¹ | 6.3 × 10⁻³ |
| Penicillin G | 3.1 × 10⁻¹ | 2.5 | 10 | 6.3 × 10⁻¹ | 10 | 6.3 × 10⁻¹ |
| FIC** | 0.12 | 1.5 | ND | 0.41 | ND | 0.41 |

*Synergy with fractional inhibition concentration (FIC) ≤ 0.5.
**Synergy with fractional inhibition concentration (FIC) ≤ 0.75.
ND: not determined. The MIC values exceeded the maximum concentration tested.

### EXAMPLE 2

### Effect of Micrococcin 1 With Garvicin KS and Penicillin on Methicillin-Resistant Staphylococcus aureus in Mouse Skin-Wound Infection Model

Materials and methods

### Bacterial Strains and Growth Conditions

Staphylococcus and Enterococcus strains were grown ON in brain heart infusion BHI broth (Oxoid) at 37° C. in aerobic conditions without shaking. For in vivo imaging of bacterial infection in mice S. aureus Xen31 (Perkin Elmer, Waltham, MA) was used. The strain was derived from the parental strain S. aureus ATCC 33591, a clinical MRSA isolated from Elmhurst Hospital in New York (Schaefler et al., 1979, Antimicrob. Agents and Chemotherap., Vol. 15(1), p. 74-80). S. aureus Xen31 possesses a stable copy of the modified Photorhabdus luminescens luxABCDE operon at a single integration site on the bacterial chromosome. Other strains were taken from our collection (LMGT, NMBU).

### Antimicrobial Agents and Formulation Vector

GarKS peptides were synthesized by Pepmic Co., LTD, China with 90-99% purity and solubilized to concentrations 1-10 mg/ml in MiliQ water. MP1 was purchased from Cayman Chemical, (Michigan, USA) with 95( )0 purity and stored at the concentration 20 mg/ml in dimethyl sulfoxide. Antibiotics were obtained from Sigma and solubilized to concentrations of 5-100 mg/ml according to supplier's instructions. All antimicrobials were stored at −20° C. until use. The final formulation for mice treatment was prepared in 5% w/v hydroxypropyl cellulose (HPC), with weight-average (Mw) ˜80000 g/mol, number-average Mn ˜10,000 g/mol (Sigma).

### Antimicrobial Activity Microtiter Plate (AAMP) Assay

Antimicrobial activity was measured using the AAMP assay (Wiegand et al., 2008, Nat. Protoc., Vol. 3(2), p 163-175). Minimum inhibitory concentration (MIC) is defined as the lowest concentration of an antimicrobial or antimicrobial combination that inhibits the visible growth (at least 50% growth inhibition) of the microorganism after 24-h incubation at 37° C. in microtitre plates in 200 μl culture.

### Synergy Assessment

Synergy testing was done with a microtiter plate checkerboard assay as previously described (Orhan et al., 2005, J. Clin. Microbiol., Vol. 43(1), p 140-143). Briefly, equal amounts of antimicrobial A were applied on microtiter plate 1 in wells A1-H1 and then diluted two-fold to wells 2-12. Similarly, equal amounts of antimicrobial B were applied on microtiter plate 2 in wells A1-A12 and diluted two-fold in wells B-H. Volumes of 50 μl of antimicrobial A from each well of microtiter plate 1 were transferred into microtiter plate 3, except for wells H1-H12. Similarly, the same volumes were transferred from microtiter plate 2 into plate 3, except for wells A1-A8. A 100 μl volume of 25 times diluted ON bacterial culture of MRSA was transferred into each well of plate 3. A 50 μl volume of pure broth was added into each well in lines H1-H12 and A1-A8 which were used to estimate MIC values of pure substances (blank).

Three-component synergy was assessed by mixing the three components (100 μg of PenG, 50 μg of GarKS and 2.5 μg of MP1) into one well of a microtiter plate, compared with activity of individual antimicrobials on the same plate. The fractional inhibition concentration (FIC) which was used to define synergy was calculated as set forth in Example 1.

### Murine Experiments

Experiments on mice were approved by the Norwegian Food Safety Authority (Oslo, Norway), application no. 18/57926. In total, 72 female BALB/cJRj mice of four weeks of age were purchased from Janvier (Le Genest-Saint-Isle, France). Two to four mice were housed per cage during the whole experiment and maintained on a 12-hour light/12-hour dark cycle with ad libitum access to water and a regular chow diet (RM1; SDS Diet, Essex, UK). Mice were acclimatized in our mouse facilities for two weeks before the start of the experiments; hence the age of mice at the start of the experiments was six weeks.

Before infection and treatment, the mice were shaved as follows: mice were anesthetized with Zoletil Forte, Rompun, Fentadon (ZRF) cocktail (containing 3.3 mg Zoletyl forte, 0.5 mg Rompun and 2.6 μg Fentanyl per 1 ml 0.9% NaCl) by intraperitoneal injection (0.1 ml ZRF/10 g body weight) and shaved on the back and flanks with an electric razor. The remaining hair was removed by hair removal cream (Veet, Reckitt Benckiser, Slough, UK) according to the manufacturer's instructions. The next day the mice were again anesthetized with ZRF cocktail (0.1 ml/10 g body weight) and two skin wounds were made on the back of every mouse with a sterile biopsy punch 6 mm in diameter (Dermal Biopsy Punch, Miltex Inc, Bethpage, NY).

Prior to infection, ON-grown S. aureus Xen31 cells were washed twice in sterile saline and then suspended in ice-cold PBS buffer. Each wound was inoculated with 10 μl of PBS containing ca 2×107 CFU of S. aureus Xen31 cells using a pipette tip. After bacterial application the mice were kept on a warm pad for 10-15 min to dry the inoculum and the wounds were then covered with a 4×5 cm Tegaderm film (3M Medical Products, St. Paul, MN, USA). Mice were then left for 24 h for the infection to establish. The day after (24 h post infection; p.i.) the mice were anesthetized with 2% isoflurane and the luminescent signal was measured by IVIS Lumina II, Perkin Elmer (2 min exposure time). The luminescent signal was quantified by the software Living Image (Perkin Elmer) from regions of interest (ROIs) around the wound and expressed as photons/second/cm²/steradian.

From this point, groups of mice were subjected to 4 different treatments, either daily or on a selected number of days dependent on the type of the regimens designed (see below). The 4 different treatments were: one treated with the bacteriocin-based Formulation (5 mg/ml GarKS, 0.1 mg/ml MP1, 5 mg/ml PenG in 5% HPC gel), one treated with formulation vehicle (5% HPC gel) as a negative control, one treated with Fucidin cream (2% fusidic acid in a cream base; LEO Pharma, Denmark) as a positive control, and one left as untreated mice. To optimize the treatment, three different regimens were performed in a consecutive manner: regimen 1 involved treatments daily from day 1 p.i. until termination at day 7 (n=29); regimen 2 involved only one treatment on day 1 p.i. and left untreated until termination (day 7) (n=27), and regimen 3 involved treatments four times, on day 1, 2, 3 and 4 p.i. and left untreated until termination (day 10) (n=16). In all cases, when treated, 40 μl per each wound of either antibacterial solutions or control substances were injected under the Tegaderm using an insulin syringe (BD SafetyGlide™; 29G needle). The bioluminescent signal, produced by S. aureus Xen31 luciferase was recorded once per day before treatments, during the entire course of the experiments. At the end of each experiment mice were euthanized by cervical dislocation.

### Statistical Analysis

Data in the graphs represent luminescent signal obtained from the analysis of individual mice. For statistical analyses and graphs R Studio (Version 1.0.15) was used.

### Results

### Search For Synergetic Antimicrobial Activities

Garvicin KS (GarKS) and micrococcin P1 (MP1) are two broad-spectrum-bacteriocins, whose MIC values vary dependent on the types of indicators used. For instance, GarKS MIC values against lactococci, listeria and enterococci are in the range of 1-20 μg/ml, while MP1 are in the range of 1-10 μg/ml (data not shown). Against S. aureus MIC-values are 32 μg/ml for GarKS and 2.5 μg/ml for MP1 (Table However, it is well known that most antimicrobials often become ineffective due to resistance development, especially when they are applied in single antimicrobial formulations. This is also true for GarKS and MP1 as can be seen in FIG. 4 with an MRSA strain—with long incubation time, resistant colonies of MRSA appeared within the inhibition zones where single antimicrobials were applied. Using a checkerboard assay synergistic effects were between GarKS and MP1. Their MIC values were reduced to 0.16 μg/ml for MP1 and 8 μg/ml for GarKS when combined (Table 5). The fractional inhibition concentration (FIC) for GarKS and MP1 combination was 0.314 (FIC values ≤0.5 are considered synergetic between two components).

In order to seek even stronger synergy, we assessed the synergistic effect between GarKS and a selected set of common and well-characterized antibiotics (Table 5). These antibiotics had very poor activity against one or several of the three MRSA strains (ATCC33591-lux, USA-300 and MRSA 43484) tested due to antibiotic resistance. For instance, all three MRSA strains were cross-resistant to ampicillin and penicillin as expected (MIC over 2500 μg/ml). They were also resistant to kanamycin (MIC over 125 μg/ml). Furthermore, the strain MRSA ATCC33591-lux is most multi-resistant as it also tolerated higher concentrations of erythromycin and streptomycin (MIC over 250 μg/ml and over 2500 μg/ml, respectively).

Using a checkerboard assay against the three MRSA strains we found no synergy or only additive effects between GarKS and Kanamycin (Kan), Erythromycin (Ery), Streptomycin (Str), Tetracycline (Tet) or Chloramphenicol (Cam). Streptomycin (Str) showed moderate synergy with GarKS only against two MRSA out of three. At the same time, moderate synergy of similar scale was observed between GarKS and Ampicillin (Amp) and Penicillin G (PenG) toward all three MRSA strains (Table 5). When combined, the MIC value of Amp or PenG was reduced at least 150 times (from over 2500 μg/ml to 16 μg/ml) while GarKS was reduced 4 times (from 32 μg/ml to 8 μg/ml). The FIC value for GarKS and PenG combination was 0.26. PenG is a relatively low-cost product compared to Amp (Wright, 1999, The penicillins, in Mayo Clinic Proceedings), and also still the drug of choice for treatment of S. pyogenes—another common case of SSTI (Bisno and Stevens, 1996, N. Engl. J. Med., Vol. 334(4), p 240-245). PenG was therefore chosen for further synergy assessment with MP1.

Using a checkerboard assay we found great synergy of MP1 with PenG: MIC value of PenG was reduced to 80-150 times (from 2500 μg/ml to 16-32 μg/ml) while the MIC of MP1 was reduced 8-16 times (from 2.5 μg/ml to 0.16-0.3 μg/ml), Table 5. The FIC value for MP1 and PenG combination was 0.07-0.13, depending on the MRSA strain.

As PenG, GarKS and MP1 appear to have synergy in dual combinations, we therefore also assessed whether we can improve the synergy further by combining all three. Indeed, stronger synergy was found in the triple combination: the MIC of PenG was reduced more than 300 times (from 2500 μg/ml to 8 μg/ml), MP1 reduced about 30 times (2.5 μg/ml to 0.08 μg/ml) and garvicin KS reduced about 8 times (from 32 μg/ml to 4 μg/ml), Table 5. Combining the three antimicrobials also prevented development of MRSA resistant colonies on agar plates at least after 72h incubation at 37° C. (FIG. 4). The FIC value for the three-component formulation was 0.16, confirming strong synergy between the antimicrobials.

In addition, we also tested a larger panel of S. aureus strains (Table 6) and two other typical skin infection pathogens, S. pseudintermedius and E. faecalis (FIG. 5). As expected, the triple combination was active against all strains tested, including those resistant to other antibiotics.

### Choosing the Vector For the Formulation

Before testing the therapeutic property of the triple combination in a murine model (see below), we searched for a suitable solvent (hereby referred to as vector) to carry the three antimicrobials. Since the GarKS peptides and especially MP1 are relatively hydrophobic molecules, it was important to look for vectors that could dissolve these peptides well and at the same time allow antimicrobial activity to be assessed. GarKS, PenG and MP1 dissolved well in white Vaseline, however the Vaseline mixture showed poor antimicrobial activity on agar plate assays (data not shown); this negative result was probably due to strong hydrophobic interactions between the molecules and Vaseline, thereby preventing diffusion of the antimicrobials.

We also tested hydroxypropyl cellulose (HPC) which is a more hydrophilic polymer and commonly used for topical and oral pharmaceutical preparations (Alvarez-Lorenzo et al., 1999, Int. J. Pharmaceutics, Vol. 180(1), p 91-103). The three antimicrobials were less soluble in HPC but, based on the solubility of the individual components, we were able to make a mixture containing 5 mg/ml of GarKS, 5 mg/ml of PenG and 0.1 mg/ml of MP1 in 5% HPC which was easy to handle. The mixture was relatively stable as it did not lose its activity after storing at +5° C. for 1 month (data not shown). Interestingly, when GarKS, MP1 and PenG were dissolved in 5% HPC, the mixture in HPC increased the activity two-fold with a final MIC for GarKS and PenG 2 μg/ml and 40 ng/ml for MP1. The mixture containing 5 mg/ml of GarKS, 5 mg/ml of PenG and 0.1 mg/ml of MP1 in 5% HPC is hereafter called the “Formulation”.

### Comparison of Our Formulation With the Commercial Fucidin Cream In Vitro

Once the Formulation was created and its stability in HPC was confirmed, it was imperative to compare it with available commercial skin antimicrobial products. We decided to use Fucidin cream (LEO Pharma, Denmark), since it is widely used as topical agent against bacterial skin infections with Fusidic acid as the active ingredient (Long, 2008, Acta Derm. Venereol., Suppl. 216, p 14-20). In terms of antimicrobial activity toward MRSA (when tested against MRSA ATCCC33591-lux applied to spots, using Fucidin cream with 1 mg Fusidic acid compared to the formulation with 250 μg GarKS, 250 μg PenG and 5 μg MP1) our Formulation appeared comparable or superior to Fucidin cream (data not shown). It is noteworthy that resistant colonies appeared within the inhibition zone of Fucidin while no such resistance development was seen with the Formulation.

### The Formulation Effectively Inhibits the Growth of MRSA in a Murine Skin Infection Model

In order to assess the therapeutic efficacy of our formulation in vivo, we developed a murine wound infection model. For this a luciferase-expressing S. aureus MRSA ATCC33591-lux (PerkinElmer) was used to monitor the therapeutic effect of the Formulation. Fucidin cream (LEO Pharma, Denmark) was again used as a positive control. In all experiments wounds on the back of each animal were made by skin punching followed by inoculation of the same amount of MRSA ATCC33591-lux (Xen31) (2×107/wound). The day when the inoculation was performed is referred to day 0. Treatment started on day 1, i.e., 24 h after infection/inoculation. Three consecutive experiments were performed to optimize the treatment regimen. In the first experiment we examined whether the Formulation had a good therapeutic effect on infected mice and whether multiple treatments might have an adverse effect on their behaviour. We treated the wounds once a day for the entire period of seven days. As expected, light emissions from luciferase activity from all mice were high prior to treatments (FIG. 6). Emissions from non-treated mice then increased strongly and more or less stabilized within three to four days. In both treated groups (Formulation and Fucidin), on the other hand, the luciferase signal declined sharply the first day after treatment (day 2) to near undetectable levels and stayed low over the entire period. Interestingly, we noticed that the signal reappeared in one of the Fucidin treated mice at day seven (FIG. 7A), indicating a possible resistance development. To confirm this a luminescent isolate from this mouse wound was rechallenged with the antimicrobials. As expected, this isolate was indeed resistant to Fucidin but not to the Formulation (FIG. 7B).

All mice showed no obvious signs of abnormal behaviour, neither in the non-treated group nor in the treated groups (vector, Fucidin and Formulation), indicating that the Formulation had no obvious toxic effects.

Given the good therapeutic effect of the Formulation, we next examined the lasting effect of one treatment. We performed the infection as described above but with only one treatment (performed 24 h after infection), followed by daily imaging sessions with no additional treatment. As can be seen in FIG. 8, for the untreated and vector-treated groups, the signals stayed relatively high compared to the positive control (Fucidin treated group). As expected, the relatively high signals at the beginning disappeared quickly in both Formulation and Fucidin treated mice at day 2, but the signal in the Formulation group reappeared again on day 4 and stayed elevated during the rest of the experiment. The Fucidin group remained low over the entire period (FIG. 8).

Based on the observations above that one treatment was not sufficient to suppress the infection for a long period, we decided to increase the number of treatments with the Formulation to four times, i.e., once a day during the first four days after the infection day. Again, we scored the emission signal over the entire period of the experiment (10 days). For comparison, we also included two control groups: one untreated and one treated with Formulation every day over the entire experiment (excepted day 10 to only score the light signals). As seen in FIG. 9, the Formulation again caused a sharp decline in luciferase signal after treatment and stayed low for at least six days after the last treatment at day 4. As expected, the same was true for the group treated daily for nine days with the Formulation. We thus concluded that a regimen of four days of treatment and once a day, was enough for creating a long-lasting antibacterial effect of the Formulation.

TABLE 5
MIC values of different antimicrobials (μg/ml) against three MRSA strains, assessed individually and in combination with bacteriocins

| | Strain | | | | | | | |
|---|---|---|---|---|---|---|---|---|
| | MRSA ATCC 33591-lux | | MRSA USA-300 | | MRSA 43484 | | All three strains* | |
| | | Comb. with | | Comb. with | | Comb. with | | Comb. with |
| | Single | GarKS | Single | GarKS | Single | GarKS | Single | GarKS |
| Antimicrobial | antimicrobial | Antibiotic/GarKS | antimicrobial | Antibiotic/GarKS | antimicrobial | Antibiotic/GarKS | antimicrobial | Antibiotic/GarKS |
| GarKS | 32 | — | 32 | — | 32 | — | 32 | — |
| MP1 | 2.5 | 0.16/8 | 2.5 | 0.16/8 | 2.5 | 0.16/8 | 2.5 | 0.16/8 |
| Amp | <2500 | 16/8 | <2500 | 16/8 | <2500 | 16/8 | <2500 | 16/8 |
| PenG | <2500 | 16/8 | <2500 | 16/8 | <2500 | 16/8 | <2500 | 16/8 |
| Kan | >125 | 32/32 | >125 | 32/32 | >125 | 32/32 | >125 | 32/32 |
| Ery | >250 | 62/32 | 31 | 16/8 | 62 | 32/16 | 31->2500 | 16-62/8-32 |
| Str | >2500 | 32/8 | 62 | 32/16 | 62 | 32/16 | 62->2500 | 32/8-16 |
| Tet | 150 | 38/32 | 1.2 | 0.6/16 | 1.2 | 0.6/16 | 1.2-150 | 0.6-37/16-32 |
| Cam | 125 | 32/32 | 125 | 64/32 | 16 | 64/32 | 16-125 | 32-64/8-32 |

| | Comb. with MP1 antibiotic/MP1 | Comb. with MP1 antibiotic/MP1 | Comb. with MP1 antibiotic/MP1 | Comb. with MP1 antibiotic/MP1 |
|---|---|---|---|---|
| PenG | 16/0.16 | 32/0.3 | 32/0.3 | 16-32/0.16-0.3 |
| | Comb. with GarKS and MP1 | Comb. with GarKS and MP1 | Comb. with GarKS and MP1 | Comb. with GarKS and MP1 |
| | antibiotic/GarKS/MP1 | antibiotic/GarKS/MP1 | antibiotic/GarKS/MP1 | antibiotic/GarKS/MP1 |
| PenG | 8/4/0.08 | 8/4/0.08 | 8/4/0.08 | 8/4/0.08 |

TABLE 6
MIC values of selected antimicrobials (μg/ml), as single components
and as a formulation mixture against different strains of S. aureus

| | Antimicrobial | | | | | | | | |
|---|---|---|---|---|---|---|---|---|---|
| | | | | | | | | | Combined activity |
| Strain | Ery | Str | Tet | Cam | Kan | GarKS | MP1 | PenG | PenG/GarKS/MP1 |
| LMG3322 | <2.5 | 62 | <0.3 | 8 | 64 | 16 | >1.8 | <0.24 | <0.24/<0.12/<0.002 |
| LMG3255 | <2.5 | 125 | 2.4 | 16 | >125 | 32 | >1.8 | <0.24 | <0.24/<0.12/<0.002 |
| LMG3271 | <2.5 | 125 | 2.4 | 16 | 64 | 32 | >1.8 | <0.24 | <0.24/<0.12/<0.002 |
| LMG3314 | <2.5 | 62 | 1.2 | 8 | 64 | 32 | 0.4 | <0.24 | <0.24/<0.12/<0.002 |
| LMG3328 | <2.5 | 62 | 1.2 | 8 | 32 | 32 | 0.9 | <0.24 | <0.24/<0.12/<0.002 |
| LMG3307 | <2.5 | 62 | 0.6 | 8 | 64 | 32 | 0.9 | <0.24 | <0.24/<0.12/<0.002 |
| LMG3242 | <2.5 | 250 | 1.2 | 8 | >125 | 32 | 0.4 | 0.24 | <0.24/<0.12/<0.002 |
| LMG3264 | <2.5 | >250 | 2.4 | 16 | >125 | 32 | >1.8 | 4 | 0.5/0.24/0.003 |
| LMG3266 | >2500 | >125 | 1.2 | 32 | >125 | 32 | >1.8 | 64 | 8/4/0.05 |
| LMG3258 | <2.5 | 62 | 1.2 | 16 | 125 | 32 | 1.8 | 64 | 8/4/0.05 |
| LMG3263 | >2500 | 31 | 2.4 | 32 | >125 | 16 | 1.8 | 125 | 8/4/0.05 |
| LMG3265 | >2500 | 31 | 2.4 | 32 | 64 | 16 | 1.8 | 125 | 8/4/0.05 |
| LMG3026 | <2.5 | 31 | 77 | 8 | 16 | 16 | >1.8 | >2500 | 4/2/0.025 |

### EXAMPLE 3: Effect of Micrococcin 1 With Various Second Bacteriocins and Antibiotics on Various Bacteria

### Materials and Methods.

### Bacterial Strains and Growth Conditions

The Staphylococcus and Enterococcus strains were grown ON in brain heart infusion BHI broth (Oxoid) at 37° C. in aerobic conditions without shaking. The S. haemoliticus strain was obtained from the Norwegian Veterinary High School (NVH) collection. The other strains were taken from our collection (LMGT, NMBU).

### Antimicrobial Agents and Formulation Vector

EntK1 and hybrid K1-EJ peptides were synthesized by Pepmic Co., LTD, China with 90-99% purity and solubilized to concentrations 1-10 mg/ml in MiliQ water. MP1 was purchased from Cayman Chemical, (Michigan, USA) with ≥95% purity and stored at the concentration 20 mg/ml in dimethyl sulfoxide. Antibiotics were obtained from Sigma and solubilized to concentrations of 5-100 mg/ml according to the supplier's instructions. All antimicrobials were stored at −20° C. until use. Farnesol, 95% purity was obtained from Sigma. Due to its high hydrophobicity, to be used in microtiter assays, it was diluted in dimethyl sulfoxide ten times and stored at −20° C. before use.

### Antimicrobial Activity Microtiter Plate (AAMP) Assay

Antimicrobial activity was measured using the AAMP assay as described in Example 2.

### Synergy Assessment

Synergy testing was performed as in Example 2.

### Results

The effects of combining micrococcin P1 with various other antimicrobials is shown in Table 7. When tested against the strains indicated combinations of farnesol/MP1, nisinZ/MP1, EntK1/MP1 and Hybrid K1-EJ/MP1 reduced MIC values significantly.

TABLE 7
MIC values of selected antimicrobials (μg/ml), as single components
and formulation mixture against different bacterial strains

| | | |
|---|---|---|
| Antimicrobial | Single | Comb. with antibiotic |
| ↓ | antimicrobial | Antibiotic/MP1 |

MRSA ATCC 33591

| | | |
|---|---|---|
| MP1 | 2.5 | — |
| Tet | 150 | 10/0.08 |
| Cam | 62 | 8/0.08 |
| Rifampicin | >100 | 1.5/0.08 |
| Fusidic acid | 0.6 | 0.04/0.15 |

Comb. Farnesol Farnesol/MP1

| | | |
|---|---|---|
| Farnesol | 56 | 14/0.08 |

Comb. nisinZ nisinZ/MP1

| | | |
|---|---|---|
| nisinZ | >12.5 | 0.8/0.3 |

| | |
|---|---|
| Single | Comb. with EntK1 |
| antimicrobial | bacteriocin/MP1 |

E. faecium LMGT 3104

| | | |
|---|---|---|
| MP1 | 2.5 | — |
| EntK1 | >250 | 0.4/0.02 |

| | |
|---|---|
| Single | Comb. with Hybrid K1-EJ |
| antimicrobial | bacteriocin/MP1 |

S. haemoliticus NVH 409

| | | |
|---|---|---|
| MP1 | 1.25 | — |
| Hybrid K1-EJ | >25 | 3/0.08 |

E.faecalis 3689

| | | |
|---|---|---|
| MP1 | >45 | — |
| Hybrid K1-EJ | >25 | 1.5/3 |

E. faecalis 3351

| | | |
|---|---|---|
| MP1 | >45 | — |
| Hybrid K1-EJ | >25 | 3/5.6 |

### EXAMPLE 4

### Effect of Micrococcin 1 With Various Antibiotics on MRSP 45 Bacteria

### Materials and Methods

### Bacterial Strains and Growth Conditions

Methicillin resistant S. pseudintermedius 45 was grown in brain heart infusion BHI broth (Oxoid, United Kingdom) at 37° C. under aerobic conditions without shaking.

### Antimicrobial Agents and Formulation Vector

Antibiotics were obtained from Sigma and solubilized to concentrations of 5-100 mg/ml according to supplier's instructions. All antimicrobials were stored at −20° C. until use.

### Antimicrobial Activity Microtiter Plate (AAMP) Assay

Antimicrobial activity was measured using the AAMP assay as described in Example 2.

### Synergy Assessment

Synergy testing was performed as in Example 2.

### Results

The effects of combining micrococcin P1 with various other antimicrobials is shown in Table 8. All combinations resulted in a significant reduction in MIC values.

TABLE 8
MIC values of selected antimicrobials (μg/ml), as single components
and formulation mixture against different bacterial strains

| | | | Fractional |
|---|---|---|---|
| | | | Inhibitory |
| | | MRSP 45 24 h | Concentration |
| | | MIC, μg/ml | (FIC) |
| | MP1 | >2.5 | |
| | Tet | 150 | |
| | Str | >2500 | |
| | Ery | >250 | |
| | CAM | 62 | |
| | Kan | >1250 | |
| | Fusidic acid | 1.25 | |
| MP1+ | Tet | 0.3/38 | 0.37 |
| MP1+ | Str | 2.5/2500 | >0.5 |
| MP1+ | Ery | >2.5/>250 | >0.5 |
| MP1+ | CAM | 0.3/32 | >0.5 |
| MP1+ | Kan | >2.5/>1250 | >0.5 |
| MP1+ | Fusidic acid | 0.6/0.02 | 0.25 |

### Example 5

### Effect of Micrococcin 1 With the Bacteriocin EJ97Short and Penicillin G on the Bacteria Methicillin-Resistant Staphylococcus pseudintermedius, MRSP45

### Materials and Methods.

### Bacterial Strains and Growth Conditions

Methicillin resistant S. pseudintermedius MRSP 45 was grown overnight in brain heart infusion BHI broth (Oxoid, United Kingdom) at 37° C. under aerobic conditions without shaking.

### Antimicrobial Agents and Formulation Vector

EJ97short peptide was synthesized by Pepmic Co., LTD, China with >95% purity and solubilized to concentrations 1-10 mg/ml in MiliQ water. MP1 was purchased from Cayman Chemical, (Michigan, USA) with ≥95% purity and stored at the concentration 20 mg/ml in dimethyl sulfoxide. Penicillin G (Sigma) was solubilized to a stock concentration of 100 mg/ml. All antimicrobials were stored at −20° C. until use.

Antimicrobial Activity Microtiter Plate (AAMP) assay

Antimicrobial activity was measured using the AAMP assay as described in Example 2.

### Synergy Assessment

Synergy testing was performed as in Example 2.

### Results

The effects of combining micrococcin P1 with EJ97short and/or PenG is shown in Table 9. When tested against the strains indicated combinations of EJ97short/MP1, PenG/MP1 and EJ97short/PenG/MP1 reduced MIC values significantly.

TABLE 9
MIC values of selected antimicrobials (μg/ml), as
single components and formulation mixture against the bacterial
strain S. pseudintermedius MRSP 45.

| Antimicrobials | MIC (μg/ml) 24 h |
|---|---|
| MP1 | 1.6->25 |
| EJ97 short | >25 |
| PenG | >125 |
| EJ97sh + MP1 | 6/0.6 |
| EJ97sh + PenG | 25/125 |
| MP1 + PenG | 0.6/32 |
| MP1 + EJ97sh + PenG | 0.16/1.6//8 |

FIC for MP1 + EJsh + PG = <0.3

### EXAMPLE 6

### Effect of Micrococcin 1 With Rifampicin or Tetracycline on Methicillin-Resistant Staphylococcus aureus in Mouse Skin-Wound Infection Model

The experiment was performed essentially as set out in Example 2, except that the following regimes as described below, were used.

Two sets of experiments were used.
A. Three mice were used and treated with the following:
   M1: tetracycline, 0.45 mg/ml
   M2: MP1, 30 μg/ml
   M3: tetracycline and MP1 with doses as above.

50-70 μl of cream was applied per wound.

Treatment was daily for 5 days (except for on day 4).
B. Seven mice were used and treated with the following:
   M1, M2: rifampicin, 150 μg/ml
   M3, M4: MP1, 8 μg/ml
   M5, M6: rifampicin+MP1, 150 μg/ml+8 μg/ml

### M7: Untreated

50-70 μl of cream was applied per wound.

Treatment was daily for 4 days.

### Results

The results are shown in FIGS. 10 and 11. It is evident from FIG. 10 that the use of the combination of MP1 and tetracycline offers a significant improvement over single antibiotic use. The co-use of rifampicin and MP1 showed synergistic effects (FIG. 11).

The rifampicin/MP1 experiment was repeated on a larger group of mice in each category (8 mice for each of rifampicin, MP1, rifampicin+MP1, 8 mice with APO base cream 30% as used for rifampicin and MP1, 7 mice untreated). Seven mice were also treated with fucidin cream (2% fusidic acid in a cream base) as a positive control. Similar results to those in FIG. 11 were observed (data not shown). In particular, rifampicin and MP1 displayed a clear synergistic effect on MRSA in the mouse skin wound model. This combination was at least as effective as fucidin. Pure rifampicin had a significant antimicrobial effect (comparable with the mixture of rifampicin and MP1) but only lasted for less than 48 hours. After that time the treatment with pure rifampicin at this concentration became inefficient (probably because of resistance development in MRSA). Neither MP1 nor the cream base showed any antimicrobial effect.

### EXAMPLE 7

### Effect of Micrococcin P1 Combined With K1-EJ and/or Garvicin KS on Planktonic or Biofilm-Associated S. haemolyticus Strains

### Materials and Methods

Bacterial strains and cultivation conditions. Bacterial strains used in this study are listed in Table 10. All strains were cultivated in Brain heart infusion (BHI) broth (Oxoid).

TABLE 10
Strains used

| S. haemolyticus | | | |
|---|---|---|---|
| strain | Isolation source (*) | ENA Acc. No. | Reference |
| 7067_4_39 | Blood culture, OUH | ERS066281 | ¹ |
| SH14 | Commensal, skin, UNN | GCA_903969855 | ¹ |
| 7067_4_60 | Blood, Switzerland | ERS066392 | ² |
| 7067_4_21 | Commensal, Japan | ERS066353 | ² |
| 7067_4_28 | Blood, OUH | ERS066270 | ² |
| 7068_7_63 | Blood, Switzerland | ERS066395 | ² |
| 4068 | Leprosy-associated | N/A | This study |
| | plantar skin ulcers, | | |
| | BPHRC | | |
| 4069 | Leprosy-associated | N/A | This study |
| | plantar skin ulcers, | | |
| | BPHRC | | |
| 4070 | Leprosy-associated | N/A | This study |
| | plantar skin ulcers, | | |
| | BPHRC | | |
| 4071 | Leprosy-associated | N/A | This study |
| | plantar skin ulcers, | | |
| | BPHRC | | |
| 4072 | Leprosy-associated | N/A | This study |
| | plantar skin ulcers, | | |
| | BPHRC | | |
| 4073 | Leprosy-associated | N/A | This study |
| | plantar skin ulcers, | | |
| | BPHRC | | |

¹Pain et al., 2019, Front Microbiol., 10, 2096
²Cavanagh et al., 2014, J. Antimicrob. Chemother., 69, 2920-2927

Bacteriocins and antimicrobial assays. K1-EJ and garvicin KS peptides (Gak-A, -B and -C) were synthesized by Pepmic Co., Ltd, China, with >95% purity. These bacteriocins were all solubilized in 0.1% (vol/vol) trifluoracetic acid (TFA; Sigma-Aldrich). Micrococcin P1 was purchased from Cayman Chemical, Michigan, USA with 95% purity and solubilized in a 50% (v/v) mixture of isopropanol (Merck) with (v/v) TFA (Sigma-Aldrich) at a stock concentration of 1 mg/ml. Cell growth inhibition assays and antimicrobial synergy determination was performed in line with Example 1.

Biofilm formation assay. Biofilms were generated using the strains 4068, 4069, 4070, 4071, 4072, 4073, 7068_7_63, 7067_4_28, 7067_4_21, 7067_4_60, SH14 and 7067_4_39. Biofilms of the S. haemolyticus strains were prepared by inoculating wells in a 96-well plate and incubating at 37° C. for 24 h. Biofilm quantification was performed using crystal violet solution.

Biofilm-oriented antimicrobial test (BOAT). The BOAT assay was performed essentially as described in Example 1. K1-EJ, garvicin KS and micrococin P1 were assessed individually or in combinations, using a serial two-fold dilution scheme of their concentration. Unless otherwise stated, the starting dilutions were 625 μg/ml for K1-EJ and garvicin KS, 62.5 μg/ml for micrococcin P1. Biofilms were allowed to form for 24 h as described above and subsequently challenged with the bacteriocins, individually or in combinations, for 5, 24 or 48 h at 37° C. As negative control, the assay was performed using the vehicles to their working concentration (i.e., without bacteriocins).

### Determination of Persister Cells to Determine Bacterial Viability Within Biofilms After BOAT.

This was performed as described in Example 1.

Confocal microscopy of biofilms. Laser scanning confocal microscopy was performed as described in Example 1.

### Results

To investigate whether there was synergy between the three bacteriocins (K1-EJ, GarKS and MP1), a checkboard antimicrobial assay was performed. The selected strains were treated for 5, 24 and 48 h with a range of concentrations of K1-EJ, GarKS and MP1 alone or in different combinations. The results are summarized in Table 11. After a 5 hour incubation, MP1 showed the strongest antimicrobial activity, with MIC₅₀ values ranging between 0.02 and 0.15 μg/ml, whereas K1-EJ had a MIC₅₀ of 0.78 μg/mI against all strains. GarKS was the least active bacteriocin, with MIC₅₀ values ranging between 3 and 24 μg/ml. The prolonged exposure for 24 and 48 h resulted in a progressive increase in the MIC₅₀ values for all treatments, a sign of resistance development. Double combinatorial treatments (GarKS/MP1; K1-EJ/GarKS; K1-EJ/MP1) increased the antimicrobial activity of the bacteriocins. Combining all three bacteriocins efficiently inhibited the microbial growth at all time-points and led to contained MIC₅₀ values, with reduced inter-strain variability and displayed no sign of resistance development. This was particularly evident at the 24 and 48 h time-points.

Most pathogenic S. haemolyticus strains are strong biofilm-formers. Given the strong antimicrobial effect of the tricomponent combination, we examined its efficacy against biofilm-associated S. haemolyticus cells using a modified version of the biofilm-oriented antimicrobial test (BOAT) which allows the quantification of the metabolic activity (via the metabolic indicator triphenyl-tetrazolium chloride) within residual live cells within the biofilms after antimicrobial treatment. To do this, the S. haemolyticus biofilms (strains 4068, 4069, 4070, 4071, 4072, 4073, 7068_7_63, 7067_4_28, 7067_4_21, 7067_4_60, SH14 and 7067_4_39) were first allowed to form for 24 h, then the biofilms were treated with the antimicrobials for 5, 24 or 48 h, before the BOAT assay was performed. We exposed biofilm-associated cells to a serial dilution of the tricomponent combination, starting with the highest concentrations (D0) being 625 μg/ml for K1-EJ and garvicin KS and 62.5 μg/ml for micrococcin P1. These high values were 100 times higher the MIC₅₀ values for planktonic cells, accounting for the higher resilience to antimicrobial treatment of biofilm-associated cells.

After 5 h the metabolic activity of all tested strains was very low or undetectable at all dilutions, except for 4069, which showed a residual metabolic activity at the highest dilution factors (data not shown). After a prolonged incubation for 24 and 48 h, most strains showed resilience but only at the lowest concentrations. For strains 4071-4073, 7068_7_63 and SH14, little or no metabolic activity was seen at all dilutions (data not shown).

Bacterial cells can remain dormant (with very low or no metabolic activity) within the biofilms. If this was the case in our biofilm settings, these cells would be overlooked by the BOAT assay. To examine this issue, biofilm-associated S. haemolyticus cells (strains 4068, 4069, 4070, 7068_7_63, 7067_4_28 and 7067_4_21) were treated with the tricomponent combination at concentration D0 (e.g. 625 μg/ml for K1-EJ and garvicin KS and 62.5 μg/ml for micrococcin P1) for 24 h. This was followed by CFU determination, confirming that the treatment led to a dramatic and statistically significant (p<0.0001) reduction of the biofilm-associated cell viability, with a drop in Log₁₀CFU values ranging between 3.3 and 3.8 compared with the non-treated control for all six S. haemolyticus strains. These results were further confirmed by LIVE/DEAD biofilm staining followed by confocal microscopy analysis (data not shown) which indeed showed only dead cells within the biofilms when treated with the tricomponent combination. On the other hand, mixtures of live and dead cells were observed when treated with the single antimicrobials.

TABLE 11
Mic values (μg/ml) determined for planktonic cells after 5, 24 or
48 hour exposure to K1-EJ, GarKS, MP1 or the indicated combinations.

| | | Strains | | | | | |
|---|---|---|---|---|---|---|---|
| Antimicrobial | | 4068 | 4069 | 4070 | 4071 | 4072 | 4073 |
| Individual component | | | | | | | |
| K1-EJ | 5 h | 0.78 | 0.78 | 0.78 | 0.78 | 0.78 | 0.78 |
| | 24 h | >100 | 0.78 | >100 | >100 | >100 | >100 |
| | 48 h | >100 | >100 | >100 | >100 | >100 | >100 |
| GarKS | 5 h | 6.5 | 3.3 | 12 | 12.5 | 23.6 | 12.5 |
| | 24 h | 24 | 13 | 48 | 51 | 49 | 51 |
| | 48 h | 25 | 26 | 51 | 100 | 52 | 51 |
| MP1 | 5 h | 0.02 | 0.15 | 0.02 | 0.043 | 0.039 | 0.022 |
| | 24 h | 0.023 | >10 | 0.33 | >10 | >10 | 0.072 |
| | 48 h | 0.078 | >10 | 0.13 | >10 | 0.63 | 0.14 |
| Combination | | | | | | | |
| K1-EJ | 5 h | 0.83 | 1.4 | 0.72 | 1.6 | 1.5 | 1.7 |
| | 24 h | 3.1 | 6 | 3.3 | 12 | 12 | 6.3 |
| | 48 h | 6 | 6.5 | 6.3 | 24 | 13 | 12 |
| GarKS | 5 h | 0.83 | 1.4 | 0.72 | 1.6 | 1.5 | 1.7 |
| | 24 h | 3.1 | 6 | 3.3 | 12 | 12 | 6.3 |
| | 48 h | 6 | 6.5 | 6.3 | 24 | 13 | 12 |
| FIC* | 5 h | 1.2 | 2.2 | 1.0 | 2.2 | 2.0 | 2.3 |
| | 24 h | 0.16 | 8.16 | 0.1 | 0.36 | 0.36 | 0.19 |
| | 48 h | 0.3 | 0.3 | 0.2 | 4.8 | 1.6 | 1.4 |
| Combination | | | | | | | |
| K1-EJ | 5 h | 0.2 | 1.5 | 0.2 | 0.42 | 0.36 | 0.2 |
| | 24 h | 0.78 | 23 | 2.8 | 1.4 | 1.5 | 0.36 |
| | 48 h | 0.72 | 48 | 6 | 3 | 1.6 | 0.85 |
| MP1 | 5 h | 0.02 | 0.15 | 0.02 | 0.042 | 0.036 | 0.02 |
| | 24 h | 0.078 | 2.3 | 0.28 | 0.14 | 0.15 | 0.036 |
| | 48 h | 0.072 | 4.8 | 0.6 | 0.3 | 0.16 | 0.01 |
| FIC* | 5 h | 0.36 | 2.0 | 0.36 | 0.64 | 0.55 | 0.36 |
| | 24 h | 0.35 | 29.7 | 0.11 | 0.028 | 0.03 | 0.05 |
| | 48 h | 0.1 | 5.3 | 0.1 | 0.06 | 0.04 | 0.01 |
| Combination | | | | | | | |
| GarKS | 5 h | 0.2 | 1.6 | 0.2 | 0.68 | 0.73 | 0.2 |
| | 24 h | 0.32 | 25 | 3.3 | 6.3 | 1.6 | 0.73 |
| | 48 h | 0.68 | 50 | 6.3 | 6.5 | 3.1 | 0.83 |
| MP1 | 5 h | 0.02 | 0.16 | 0.02 | 0.068 | 0.073 | 0.02 |
| | 24 h | 0.032 | 2.5 | 0.33 | 0.63 | 0.16 | 0.073 |
| | 48 h | 0.068 | 5 | 0.63 | 0.65 | 0.31 | 0.083 |
| FIC* | 5 h | 0.13 | 0.6 | 0.12 | 0.06 | 0.2 | 0.1 |
| | 24 h | 0.15 | 2.2 | 0.17 | 0.19 | 0.05 | 0.12 |
| | 48 h | 0.12 | 1.3 | 0.2 | 0.13 | 0.1 | 0.7 |
| Combination | | | | | | | |
| K1-EJ | 5 h | 0.32 | 0.68 | 0.32 | 0.72 | 0.68 | 0.33 |
| | 24 h | 0.78 | 3.3 | 2.8 | 1.6 | 1.6 | 0.78 |
| | 48 h | 2.8 | 5.8 | 5.8 | 3.3 | 5.8 | 1.6 |
| GarKS | 5 h | 0.32 | 0.68 | 0.32 | 0.72 | 0.68 | 0.33 |
| | 24 h | 0.78 | 3.3 | 2.8 | 1.6 | 1.6 | 0.78 |
| | 48 h | 2.8 | 5.8 | 5.8 | 3.3 | 5.8 | 1.6 |
| MP1 | 5 h | 0.032 | 0.068 | 0.032 | 0.072 | 0.068 | 0.033 |
| | 24 h | 0.078 | 0.33 | 0.28 | 0.16 | 0.16 | 0.078 |
| | 48 h | 0.28 | 0.58 | 0.58 | 0.33 | 0.58 | 0.16 |
| FIC** | 5 h | 0.62 | 1.12 | 0.6 | 1.2 | 1.1 | 0.6 |
| | 24 h | 0.38 | 4.5 | 0.17 | 0.06 | 0.06 | 0.14 |
| | 48 h | 0.5 | 0.4 | 0.2 | 0.1 | 0.26 | 0.15 |

*Synergy with fractional inhibition concentration (FIC) ≤ 0.5.
**Synergy with fractional inhibition concentration (FIC) ≤ 0.75.

1. An antibacterial composition comprising micrococcin P1 and at least one additional antibacterial agent, wherein preferably said additional antibacterial agent is not a thiopeptide.

2. An antibacterial composition as claimed in claim 1 wherein the at least one antibacterial agent is selected from:
i) at least one second bacteriocin; and/or
ii) at least one antibiotic.

3. An antibacterial composition as claimed in claim 1 or 2, wherein said at least one second bacteriocin is selected from:
i) a multi-peptide bacteriocin complex, preferably a di- or tri-peptide bacteriocin complex, and/or
ii) a peptide comprising an amino acid sequence selected from:
a)
(EntK1, SEQ ID NO: 1)
MKFKFNPTGTIVKKLTQYEIAWFKNKHGYYPWEIPRC
or
(EntEJ97, SEQ ID NO: 2)
MLAKIKAMIKKFPNPYTLAAKLTTYEINWYKQQYGRYPWERPVA,
b) a sequence with at least 40% sequence identity to sequence a),
c) a sequence consisting of at least 15 consecutive amino acids of sequence a), and
d) a sequence with at least 40% sequence identity to sequence c),
wherein sequences b), c) and d) comprise at least the consensus sequence KXXXGXXPWE, wherein X may be any amino acid.

4. An antibacterial composition as claimed in claim 3 wherein said multi-peptide bacteriocin complex comprises two or more peptides selected from:
a) a peptide comprising the sequence as set forth in SEQ ID NO:6 (GarA) or a sequence with at least 50% sequence identity thereto;
b) a peptide comprising the sequence as set forth in SEQ ID NO:7 (GarB) or a sequence with at least sequence identity thereto; and
c) a peptide comprising the sequence as set forth in SEQ ID NO:8 (GarC) or a sequence with at least 50% sequence identity thereto;
wherein the sequence as set forth in SEQ ID NO:6 or said sequence with at least 50% sequence identity thereto comprises at least two tryptophan residues, wherein said complex has antibacterial activity.

5. An antibacterial composition as claimed in claim 3 wherein the second bacteriocin is a peptide which comprises or consists of
MKFKFNPTGTIVKKLTQYEIAWFKNKHGYYPWEIPRC (EntK1), or
MKFKFNPTGTIVKKLTQYEINWYKQQYGRYPWERPVA (K1-EJ hybrid), or a sequence with at least 50%, preferably 80% sequence identity thereto.

6. An antibacterial composition as claimed in claim 3 wherein the second bacteriocin is a peptide which comprises or consists of
MIKKFPNPYTLAAKLTTYEINWYKQQYGRYPWERPVA or a sequence with at least 50%, preferably 80% sequence identity thereto (preferably
MIKKFPNPYTLAAKLTTYEINWYKQQYGRYPWERPVA, SEQ ID NO:3, EntEJ97short).

7. An antibacterial composition as claimed in any one of claims 2 to 6 wherein the antibiotic is selected from one or more of chloramphenicol, a β-lactam antibiotic (preferably a penicillin, preferably penicillin G), rifampicin, fusidic acid, tetracycline, streptomycin, erythromycin, kanamycin and lantibiotic nisin.

8. An antibacterial composition as claimed in any one of claims 1 to 7 wherein the antibacterial agent is farnesol.

9. An antibacterial composition as claimed in any one of claims 1 to 8 wherein said composition comprises more than one additional antibacterial agent.

10. An antibacterial composition as claimed in any one of claims 1 to 9 wherein said composition comprises:
i) MP1 and Garvicin KS or a related molecule as defined in claim 4;
ii) MP1 and an essential oil compound, preferably farnesol;
iii) MP1 and a tetracycline, preferably tetracycline;
iv) MP1 and an amphenicol, preferably chloramphenicol;
v) MP1 and rifampicin;
vi) MP1 and fusidic acid;
vii) MP1 and an aminoglycoside, preferably streptomycin or kanamycin;
viii) MP1 and a macrolide, preferably erythromycin;
ix) MP1 and a lantibiotic, preferably nisin;
x) MP1 and EntK1 or a related molecule as defined in claim 3, preferably a K1-EJ hybrid or a sequence with at least 80% sequence identity thereto as defined in claim 5, and optionally Garvicin KS or a related molecule as defined in claim 4;
xi) MP1 and a 8-lactam antibiotic (preferably penicillin G);
xii) MP1, Garvicin KS ora related molecule as defined in claim 4, and a β-lactam antibiotic (preferably penicillin G);
xiii) MP1, EntK1 or a related molecule as defined in claim 3, preferably EJ97short or a related molecule as defined in claim 6, and optionally a β-lactam antibiotic (preferably penicillin G).

11. A composition as claimed in any one of claims 1 to 10, wherein said composition has antibacterial activity against at least one bacteria selected from the genera Bacillus, Streptococcus, Listeria, Enterococcus, Staphylococcus, Acinetobacter and Paenibacillus, preferably selected from the species Bacillus cereus, Listeria monocytogenes, Listeria innocua, Listeria grayi, Listeria seelingeri, Streptococcus thermophylus, Streptococcus agalactia, Streptococcus pneumonia, Streptococcus salivarius, Enterococcus faecalis, Enterococcus faecium, Staphylococcus aureus, Staphylococcus hemolyticus, Staphylococcus pseudintermedius, Acinetobacter nosocomialis and Paenibacillus larvae, particularly preferably Methicillin-resistant Staphylococcus aureus (MRSA), Methicillin-resistant Staphylococcus pseudintermedius (MRSP), Vancomycin-resistant Enterococci (VRE) and antibiotic-resistant strains of Listeria monocytogenes, wherein preferably said composition has antibacterial activity against at least one bacteria from each of the genera Bacillus, Streptococcus, Listeria, Enterococcus, Staphylococcus, Acinetobacter and Paenibacillus.

12. A pharmaceutical composition comprising a composition as defined in any one of claims 1 to 11 and a pharmaceutically acceptable diluent, excipient or carrier.

13. A composition as defined in any one of claims 1 to 12, or MP1 and at least one additional antibacterial agent as defined in any one of claims 1 to 12, for use in therapy.

14. A composition as defined in any one of claims 1 to 12, or MP1 and at least one additional antibacterial agent as defined in any one of claims 1 to 12, for use in treating or preventing a bacterial infection in a subject or patient, wherein preferably said bacterial infection is a skin infection (preferably caused by Staphylococcus, preferably by S. aureus, S. hemolyticus or S. pseudintermedius, or by Enterococcus, preferably by E. faecium or E. faecalis), an oral or throat infection (preferably caused by Streptococcus), an infection present in or causing dental caries (preferably caused by Streptococcus) or mastitis (preferably caused by Staphylococcus or Streptococcus), wherein preferably said subject or patient is a mammalian animal, preferably a human.

15. A composition, or MP1 and at least one additional antibacterial agent, for the use as claimed in claim 14 wherein the bacteria causing said bacterial infection is in the form of a biofilm.

16. A composition, or MP1 and at least one additional antibacterial agent, for the use as claimed in claim 14 or 15 wherein said composition is to be administered topically.

17. Use of a composition as defined in any one of claims 1 to 12, or MP1 and at least one additional antibacterial agent as defined in any one of claims 1 to 12, in the preparation of a medicament for treating or preventing a bacterial infection in a subject or patient, wherein preferably said bacterial infection is as defined in claim 14 or 15, wherein preferably said medicament is to be administered topically, wherein preferably said subject or patient is a mammalian animal, preferably a human.

18. A method of treating or preventing a bacterial infection comprising administering a composition as defined in any one of claims 1 to 12, or MP1 and at least one additional antibacterial agent as defined in any one of claims 1 to 12, to a subject or patient or a part of said subject's or patient's body, wherein preferably said bacterial infection is as defined in claim 14 or 15, wherein preferably said administration is topical, wherein preferably said subject or patient is a mammalian animal, preferably a human.

19. Use of a composition as defined in any one of claims 1 to 12, or MP1 and at least one additional antibacterial agent as defined in any one of claims 1 to 12, as an antibacterial.

20. Use of micrococcin P1 as an adjuvant in an antibacterial composition.

21. A composition, MP1 and at least one additional antibacterial agent, method or use as claimed in any one of claims 14 to 20 wherein said bacterial infection, or the bacterial infection against which the antibacterial is effective, is caused by at least one bacteria selected from the genera Bacillus, Streptococcus, Listeria, Enterococcus, Staphylococcus, Acinetobacter and Paenibacillus, preferably selected from the species Bacillus cereus, Listeria monocytogenes, Listeria innocua, Listeria grayi, Listeria seelingeri, Streptococcus thermophylus, Streptococcus agalactia, Streptococcus pneumonia, Streptococcus salivarius, Enterococcus faecalis, Enterococcus faecium, Staphylococcus aureus, Staphylococcus hemolyticus, Staphylococcus pseudintermedius, Acinetobacter nosocomialis and Paenibacillus larvae, particularly preferably Methicillin-resistant Staphylococcus aureus (MRSA), Methicillin-resistant Staphylococcus pseudintermedius (MRSP), Vancomycin-resistant Enterococci (VRE) and antibiotic-resistant strains of Listeria monocytogenes.

22. An in vitro method of killing, damaging or preventing the replication of bacteria comprising administering a composition as defined in any one of claims 1 to 12, or MP1 and at least one additional antibacterial agent as defined in any one of claims 1 to 12.

23. An in vitro method as claimed in claim 22, wherein the bacteria is in the form of a biofilm.


SEQUENCE LISTING (34 sequences)

SEQ ID NO: 1 - protein, 37 aa, Enterococcus sp.
MKFKFNPTGTIVKKLTQYEIAWFKNKHGYYPWEIPRC

SEQ ID NO: 2 - protein, 44 aa, Enterococcus sp.
MLAKIKAMIKKFPNPYTLAAKLTTYEINWYKQQYGRYPWERPVA

SEQ ID NO: 3 - protein, 37 aa, Artificial Sequence
  EntEJ97short
MIKKFPNPYTLAAKLTTYEINWYKQQYGRYPWERPVA

SEQ ID NO: 4 - protein, 37 aa, Artificial Sequence
  K1-EJ hybrid
MKFKFNPTGTIVKKLTQYEINWYKQQYGRYPWERPVA

SEQ ID NO: 5 - protein, 44 aa, Artificial Sequence
  EJ-K1 hybrid
MLAKIKAMIKKFPNPYTLAAKLTTYEIAWFKNKHGYYPWEIPRC

SEQ ID NO: 6 - protein, 34 aa, Lactococcus garvieae
MGAIIKAGAKIVGKGVLGGGASWLGWNVGEKIWK

SEQ ID NO: 7 - protein, 34 aa, Lactococcus garvieae
MGAIIKAGAKIIGKGLLGGAAGGATYGGLKKIFG

SEQ ID NO: 8 - protein, 32 aa, Lactococcus garvieae
MGAIIKAGAKIVGKGALTGGGVWLAEKLFGGK

SEQ ID NO: 9 - protein, 26 aa, Bacillus cereus
MAKIGKWVVKGAAGYLGWEIGEGIWK

SEQ ID NO: 10 - protein, 30 aa, Bacillus cereus
MGALVKGGLKLIGGTAASWLGWEAGERVWK

SEQ ID NO: 11 - protein, 30 aa, Bacillus cereus
MGAIIKGGLKLVGGGAAGGFTYGGLKKIFG

SEQ ID NO: 12 - protein, 30 aa, Bacillus cereus
MGAIIKGAAKVLGKGAATGGVIYGLEKLFR

SEQ ID NO: 13 - protein, 30 aa, Bacillus cereus
MGAVVKGGLKIIGGTAASWLGWEAGTRIWK

SEQ ID NO: 14 - protein, 30 aa, Bacillus cereus
MGAAVKMLGKAFAGGVAGGATYGGLKKIFG

SEQ ID NO: 15 - protein, 31 aa, Bacillus cereus
MGAVVKGALKIIGGGAASGGAVYGLERIFGR

SEQ ID NO: 16 - protein, 27 aa, Bacillus cereus
MGKKIGKWIITGAAGWAGWEIGEGIWK

SEQ ID NO: 17 - protein, 30 aa, Bacillus cereus
MGALFKAALKAAGGGAAGGATYGGLKHFFG

SEQ ID NO: 18 - protein, 29 aa, Bacillus cereus
MKYLGTLIKGAAGGAGAYVGEKIYNWYKN

SEQ ID NO: 19 - protein, 31 aa, Staphylococcus aureus
MGKLAIKAGKIIGGGIASALGWAAGEKAVGK

SEQ ID NO: 20 - protein, 30 aa, Staphylococcus aureus
MGAVAKFLGKAALGGAAGGATYAGLKKIFG

SEQ ID NO: 21 - protein, 31 aa, Staphylococcus aureus
MGALIKTGAKIIGSGAAGGLGTYIGHKILGK

SEQ ID NO: 22 - protein, 31 aa, Staphylococcus aureus
MGAVIKVGAKVIGWGAASGAGLYGLEKILKK

SEQ ID NO: 23 - protein, 10 aa, Artificial Sequence
  Consensus sequence
  misc_feature (2)..(4): Xaa can be any naturally occurring amino acid
  misc_feature (6)..(7): Xaa can be any naturally occurring amino acid
KXXXGXXPWE

SEQ ID NO: 24 - protein, 10 aa, Enterococcus sp.
KNKHGYYPWE

SEQ ID NO: 25 - protein, 10 aa, Enterococcus sp.
KQQYGRYPWE

SEQ ID NO: 26 - protein, 10 aa, Artificial Sequence
  Preferred consensus sequence
  MISC_FEATURE (2)..(2): Xaa is Asn or Gln
  MISC_FEATURE (3)..(3): Xaa is Lys or Gln
  MISC_FEATURE (4)..(4): Xaa is His or Tyr
  MISC_FEATURE (6)..(6): Xaa is Tyr or Arg
  MISC_FEATURE (7)..(7): Xaa is Tyr
KXXXGXXPWE

SEQ ID NO: 27 - protein, 12 aa, Artificial Sequence
  Consensus sequence
  misc_feature (1)..(2): Xaa can be any naturally occurring amino acid
  misc_feature (5)..(6): Xaa can be any naturally occurring amino acid
  misc_feature (8)..(11): Xaa can be any naturally occurring amino acid
XXGWXXGXXXXK

SEQ ID NO: 28 - protein, 12 aa, Artificial Sequence
  Preferred consensus sequence 1
  misc_feature (2)..(2): Xaa can be any naturally occurring amino acid
  misc_feature (5)..(6): Xaa can be any naturally occurring amino acid
  misc_feature (8)..(11): Xaa can be any naturally occurring amino acid
WXGWXXGXXXXK

SEQ ID NO: 29 - protein, 12 aa, Artificial Sequence
  Preferred consensus sequence 2
  misc_feature (1)..(2): Xaa can be any naturally occurring amino acid
  misc_feature (5)..(6): Xaa can be any naturally occurring amino acid
  misc_feature (8)..(10): Xaa can be any naturally occurring amino acid
XXGWXXGXXXWK

SEQ ID NO: 30 - protein, 12 aa, Artificial Sequence
  Preferred consensus sequence 3
  misc_feature (2)..(2): Xaa can be any naturally occurring amino acid
  misc_feature (5)..(6): Xaa can be any naturally occurring amino acid
  misc_feature (8)..(10): Xaa can be any naturally occurring amino acid
WXGWXXGXXXWK

SEQ ID NO: 31 - protein, 15 aa, Artificial Sequence
  Consensus sequence
  misc_feature (1)..(5): Xaa can be any naturally occurring amino acid
  misc_feature (8)..(9): Xaa can be any naturally occurring amino acid
  misc_feature (11)..(14): Xaa can be any naturally occurring amino acid
XXXXXGWXXGXXXXK

SEQ ID NO: 32 - protein, 15 aa, Artificial Sequence
  Preferred consensus sequence 1
  misc_feature (1)..(1): Xaa can be any naturally occurring amino acid
  misc_feature (3)..(5): Xaa can be any naturally occurring amino acid
  misc_feature (8)..(9): Xaa can be any naturally occurring amino acid
  misc_feature (11)..(13): Xaa can be any naturally occurring amino acid
XAXXXGWXXGXXXWK

SEQ ID NO: 33 - protein, 15 aa, Artificial Sequence
  Preferred consensus sequence 2
  misc_feature (1)..(1): Xaa can be any naturally occurring amino acid
  misc_feature (3)..(5): Xaa can be any naturally occurring amino acid
  misc_feature (8)..(9): Xaa can be any naturally occurring amino acid
  misc_feature (11)..(12): Xaa can be any naturally occurring amino acid
XAXXXGWXXGXXIWK

SEQ ID NO: 34 - protein, 15 aa, Artificial Sequence
  Preferred consensus sequence 3
  misc_feature (3)..(3): Xaa can be any naturally occurring amino acid
  misc_feature (5)..(5): Xaa can be any naturally occurring amino acid
  misc_feature (9)..(9): Xaa can be any naturally occurring amino acid
  misc_feature (12)..(12): Xaa can be any naturally occurring amino acid
AAXWXGWEXGEXIWK